(19) United States
(12) Patent Application Publication
(10) Pub. No.: US 2017/0299589 A1
(43) Pub. Date: Oct. 19, 2017

(54) Methods to Diagnose and Treat Multiple Sclerosis via Detection of Altered Protein Components of Serum

(71) Applicant: YUSCIENCE, LLC, Aurora, CO (US)

(72) Inventor: Xiaoli YU, Denver, CO (US)

(21) Appl. No.: 15/510,206

(22) PCT Filed: Sep. 8, 2015
(86) PCT No.: PCT/US2015/048935

§ 371 Date: Mar. 9, 2017

Related U.S. Application Data
(60) Provisional application No. 62/107,497, filed on Jan. 26, 2015.
(60) Provisional application No. 62/048,229, filed on Sep. 9, 2014.
(60) Provisional application No. 62/048,042, filed on Sep. 9, 2014.

(51) Int. Cl. G01N 33/564; G01N 33/68

(52) U.S. Cl. CPC G01N 33/564; G01N 33/6827; G01N 2800/285; G01N 2333/765; G01N 2800/52; G01N 2440/38

(57) ABSTRACT

The methods disclosed herein include diagnosing a patient with MS, selecting a patient for further testing for MS, should the patient show elevated level of human IgG relative to an appropriate control. The methods also include differentiating subtypes of MS. The methods also include evaluating the efficacy of an MS drug or course of drug treatments, and/or treating MS. The methods include determining whether patients have elevated levels of IgG3-IgG1 immune complexes (which can include glycosylated IgG antibodies) in both blood and CSF. Methods also include diagnosing patients with primary-progressive MS (PPMS) and secondary-progressive MS (SPMS) where patients have higher levels of IgG3-IgG1 complexes in both CSF and blood, and reduced levels of albumin compared to patients with relapsing-remitting MS (RRMS). The methods optionally include treating the sample to dissociate immune and/or protein complexes, contacting the sample with a reagent that binds specifically to a human IgG or other protein, comparing the results to an appropriate control, and determining whether the patient has an altered level of IgG or other protein consistent with MS.

### RELATED APPLICATIONS

This application claims priority to U.S. Ser. No. 62/048,229, filed Sep. 9, 2014, entitled “IgG3-IgG1 Complexes and Oligoclonal Bands in Multiple Sclerosis Diagnostics and Therapeutics”; U.S. Ser. No. 62/048,042, filed Sep. 9, 2014, entitled “Blood Assays for Early Diagnosis of Multiple Sclerosis”; and U.S. Ser. No. 62/107,497, filed Jan. 26, 2015, entitled “Serum Immunoglobulin G Antibodies in Multiple Sclerosis are elevated and Contain Partially Blocked FC Region,” each of which is incorporated herein by reference in its entirety.

### TECHNICAL FIELD

The instant invention is related to methods to diagnose and treat Multiple Sclerosis (MS).

### BACKGROUND

Multiple sclerosis (MS) is a chronic inflammatory disease of the central nervous system (CNS) with demyelination and neuronal damage, and is the most common cause of neurological disability in young adults, affecting over 2 million people worldwide. A characteristic feature of the CNS inflammatory response in MS is the increased intrathecal synthesis of immunoglobulin G (IgG), and the presence of oligoclonal bands (OCBs) in the brain and cerebral spinal fluid (CSF). More than 95% of MS patients have OCBs, providing powerful evidence for the diagnosis of MS. OCBs are defined as two or more IgG bands detected in CSF but not in corresponding serum within the same patient. MS patients with the most malignant courses of disease had raised CSF IgG levels more frequently than that of patients with a benign course. The IgG index [(CSF IgG/serum IgG)/(CSF albumin/serum albumin)] is elevated (>0.6) in about 70%-90% of MS patients, and is reduced to normal values with steroid therapy, immunomodulation or immunosuppression. OCBs are associated with a more rapid conversion from clinically isolated syndrome to clinically definite MS, and MS patients positive for OCBs have greater brain atrophy, providing evidence that they may reflect more active CNS-directed autoimmunity and contribute to tissue damage. Furthermore, when treated with natalizumab, a peripherally acting anti-VLA 4 monoclonal antibody that blocks immune cell migration into the CNS and effectively reduces MS disease activity, CSF IgG levels decrease and OCBs disappear. A recent study showed that B cells in peripheral blood participate in OCB production, suggesting the importance of serum contribution to OCB. Current laboratory diagnosis of MS relies entirely on the demonstration of elevated total IgG levels and the presence of oligoclonal bands in the CSF. The standard method to detect OCBs in MS is isoelectric focusing (IEF) followed by immunoblotting. Healthy individuals do not have intrathecal IgG, and the specificity of OCBs in MS is unknown.

Intrathecal synthesis of IgG is mainly consisted of IgG1 and IgG3 isotypes. Once present, the intrathecal synthesis of IgG, mainly consisted of IgG1 and IgG3 (Greve et al. 2001), remains stable over time. The elevation of IgG1 and IgG3 indices in MS was found more frequently (in about 90% of patients) than the elevation of the general IgG index (in 72% of patients).

A main advantage of Immunoabsorption therapy (IA) is the selective elimination of pathogenic antibodies while sparing other plasma proteins. Highly effective IA treatments were reported in patients with steroid-refractory MS.

Predicting whether patients will progress to disability—and how they will respond to treatments—is among the greatest challenges for neurologists treating MS. There are multiple medications for MS now, and it is necessarily clear to physicians which drugs to use when. For example, a patient predicted to debilitate rapidly might be treated more aggressively than one who will maintain capabilities for years to come. But the current tools fall short of making that projection accurately. The best biomarkers available today for MS, are pathological features, such as active lesions, tracked by magnetic resonance imaging (MRI). However, MRI results do not predict future disease course well, and further is expensive and impractical for frequent clinical use.

In addition, although the number of available treatments for MS has increased, little is known about surrogates that predict treatment response in individual patients.

Furthermore, analogous to cancer therapy, a potential successful treatment strategy in MS is combinatorial therapy using different drugs targeting different pathophysiological processes. Such a differential therapeutic approach requires the development of biomarkers that a) reflect the targeted immunopathological process, b) select patients in which the pathogenic process predominates, c) indicate responses to therapeutic interventions, and d) provide a simple and less expensive monitoring tool in clinical trials and routine patient management.

From a patient's perspective, what is needed is a test for MS that eliminates the need to subject the patient to the severe discomfort of lumbar punctures, the anxiety of false positive diagnoses, and remedies the difficulty of prognosis for disease progression. From the clinician's perspective, what is needed is a far more simplified, accurate, quantitative, and less costly blood test that can be routinely performed in the laboratory, as well as an assay to separate different phenotypes of MS (Progressive MS from Relapsing-Remitting MS), and a method to determine the patient's response to the increasingly expensive, diverse therapeutic modalities and ever expanding disease modified therapies (at least 12 drugs currently available). This invention serves these very important needs as will become apparent in the following disclosure.

The present invention is directed toward overcoming one or more of the problems discussed above.

### SUMMARY OF THE EMBODIMENTS

The methods disclosed herein include diagnosing a patient with MS, selecting a patient for further testing for MS, evaluating the efficacy of an MS drug or course of drug treatments, and/or treating MS. The methods include determining whether patients have elevated levels of IgG3-IgG1 immune complexes (which can include glycosylated IgG antibodies) in both blood and CSF. Methods also include diagnosing patients with primary-progressive MS (PPMS) and secondary-progressive MS (SPMS) where patients have higher levels of IgG3-IgG1 complexes in both CSF and blood, and reduced levels of albumin compared to patients with relapsing-remitting MS (RRMS). The methods optionally include treating the sample to purify and/or dissociate immune and/or protein complexes, contacting the sample with a reagent that binds specifically to a human IgG or other protein, comparing the results to an appropriate control, and determining whether the patient has an altered level of IgG or other protein consistent with MS.

Various modifications and additions can be made to the embodiments discussed without departing from the scope of the invention. For example, while the embodiments described above refer to particular features, the scope of this invention also included embodiments having different combination of features and embodiments that do not include all of the above described features.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 (a) shows a photograph of MS and healthy control sera stored in Eppendorf tubes long term at 4° C. demonstrating the protein precipitation in MS serum.

FIG. 1 (b) shows a nondenaturing (Native) PAGE of MS sera compared to healthy and CNS inflammatory control sera showing increased amounts of high molecular weight proteins in MS sera.

FIG. 1 (c) shows reduced, denaturing PAGE of MS sera compared to healthy and CNS inflammatory control sera showing higher levels of protein in MS sera.

FIG. 2 shows higher protein levels detected by BCA assay in MS sera compared to controls.

FIG. 3 shows higher protein levels detected by BSA assay in MS sera treated with borate buffer at pH 10.8 to disrupt protein complexes compared to water treatment, comparing to controls.

FIG. 4 shows an increase in 150 kD molecular weight band in MS sera after heated water treatment compared to controls when probed with anti-human albumin on Western blots SDS PAGE.

FIG. 5 (a) shows native-PAGE gel and Western Blot of MS sera compared to controls, probed with anti-IgG (H+L).

FIG. 5 (b) shows native-PAGE gel and Western Blot of MS sera compared to controls, probed with anti-human IgG-Fc.

FIG. 5 (c) shows Dot Blots of MS and healthy control sera probed with anti-human IgG (H+L)-HRP which demonstrates that serum IgG levels in MS are 1.6 times higher than that of controls.

FIG. 6 shows a flow chart for an ELISA format for measuring IgG from sera.

FIG. 7 shows elevated levels of IgG in supernatant after binding to Protein A plates (Protein A flow through) (right FIG.), and no difference on original sera bound to Protein A plates (left FIG.).

FIG. 8 shows that measurement by anti-human IgG (H+L)-HRP, there were significantly elevated amounts of IgG antibodies present in the flow-through after Protein G purification of MS sera.

FIG. 9 (a) shows higher levels of IgG1 in MS serum in an SDS PAGE Western Blot probed with anti-human IgG1.

FIG. 9 (b) shows higher levels of IgG1 in acid-treated MS serum in an SDS PAGE Western Blot probed with anti-human IgG1.

FIG. 9 (c) shows higher levels of IgG1 are detected in Protein A flow-through MS sera.

FIG. 10 shows data summary from slot blots (nondenaturing conditions, probed with anti-human IgG (H+L) antibody-HRP) demonstrating less total IgG detected in MS in both CSF and serum compared to inflammatory controls.

FIG. 11 (a) shows SDS-PAGE Western blots of 2% PEG precipitates of IgG immune complexes in MS and HC sera, probed with anti-human IgG1 antibody.

FIG. 11 (b) shows SDS-PAGE Western blots of 2% PEG precipitates IgG immune complexes in MS and HC sera, probed with anti-human IgG3 antibody.

FIG. 12 shows higher levels of IgG1 in MS sera by SDS-PAGE Western blot of 2% PEG precipitates probed with anti-human IgG1 antibody.

FIG. 13 shows higher levels of IgG3 in MS sera by SDS-PAGE Western blots probed with anti-human IgG3 after borate buffer treatment

FIG. 14 (a) shows MS total CSF and serum SDS PAGE Western blots, probed with anti-human IgG (H+L)-HRP (left), anti-IgG1 (middle) and anti-IgG3 antibodies (right), highlight IgG3 and IgG1 heavy chains in rectangle.

FIG. 14 (b) shows a summary of Western blots of significantly increased ratio of IgG3 reactive antibodies between CSF and serum compared to the ratios of IgG1 and total IgG, indicating the increased IgG in MS is due to IgG3.

FIG. 15 (a) shows a Western blot of MS and IC CSF probed with anti-human IgG1 antibody demonstrating that there is no difference in IgG1 levels between MS and IC CSF.

FIG. 15 (b) shows the same blot of FIG. 15 (a) that was stripped, and re-probed with anti-human IgG3 antibody demonstrating higher levels of IgG3 in MS CSF. Lane 1. SPMS; Lane 2. RRMS; Lane 3. IC 06-1 (SSPE); Lane 4. IC 07-2 (chronic progressive meningoencephalitis); Lane 5. IC 06-5 (paraneoplastic encephalitis); and Lane 6. IC 05-2 (Chronic meningitis).

FIG. 15 (c) shows a SDS PAGE Western blot of non-inflammatory control CSF and paired serum probed with anti-human IgG3 antibody showing no IgG3 detected in the CSF.

FIG. 16 (a) shows SDS-PAGE of MS original and purified serum by Protein A DYNABEADS, probed with anti-human IgG antibody demonstrating the depletion of IgG in MS by Protein A.

FIG. 16 (b) shows SDS-PAGE Western blots of MS original and purified serum by Protein A DYNABEADs, probed with the three most common biotinylated lectins (GSA, SNA, RCA) for determining the specificity of respective sugars on the IgG antibodies, demonstrating the depletion of glycosylated IgG in MS serum after Protein A purification.

FIG. 17 (a) shows Western blots of MS original and Protein G purified serum IgG (paired) probed with anti-human IgG Fc specific antibody-AP.

FIG. 17 (b) shows Western blots of MS original and Protein G purified serum IgG (paired) probed with anti-human IgG (H+L)-AP.

FIG. 18 shows data summary of SDS PAGE Western blots of Protein A purified IgG in CSF and paired serum from 12 MS patients and 9 inflammatory CNS controls probed with SNA lectin for sialic acid level. Significantly higher levels of sialylated IgG Fc are detected in MS serum compared to CSF.

FIG. 19 shows the results of the complement dependent cytotoxicity of MS sera and healthy sera from Protein G flow-through on mouse CNS cells.

FIG. 20 (a) shows a SDS PAGE Western Blot of sera probed with total IgG (H+L)-HRP demonstrating that SPMS have lower levels of IgG light chains (between 25 kD-50 kD) compared to RRMS.

FIG. 20 (b) shows SDS PAGE Western Blot of sera probed with anti-human albumin demonstrating the reduced levels of albumin in both SPMS and PPMS compared to RRMS.

FIG. 21 shows a comparison of PPMS and RRMS CSF by SDS PAGE Western Blot probed with anti-human IgG (H+L) antibody-HRP, demonstrating the reduced levels of IgG heavy and light chain, and increased levels of IgG3 heavy chain (65 kD) in PPMS.

FIG. 22 , shows SDS-PAGE Western blots of various sample preparations from CSF and serum of one PPMS patient (PPMS1) probed with anti-IgG3 or anti-human IgG (H+L) antibodies, demonstrating that IgG3 are most abundant IgG in total and A-, G-FT in both CSF and serum.

FIG. 23 shows SDS-PAGE Western blots of a second PPMS patient (PPMS2), probed with anti-human IgG3 antibody, demonstrating that IgG3 are most abundant IgG in total and A-, G-FT in both CSF and serum.

### DETAILED DESCRIPTION

While various aspects and features of certain embodiments have been summarized above, the following detailed description illustrates a few embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. Several embodiments are described and claimed herein, and while various features are ascribed to different embodiments, it should be appreciated that the features described with respect to one embodiment may be incorporated with other embodiments as well. By the same token, however, no single feature or features of any described or claimed embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

The present invention takes advantage of the relationship between IgG3-IgG1 immune complexes and OCBs in MS. We have shown that 1) MS patients have significantly elevated amounts of glycosylated IgG antibodies which form IgG3-IgG1 immune complexes in both blood and CSF; 2) the IgG3 subclass is responsible for the increased levels of intrathecal IgG in the CSF, 3) patients with primary-progressive MS (PPMS) have elevated levels of IgG3 in both CSF and blood, and reduced levels of albumin, compared to patients with relapsing-remitting MS (RRMS).

We have thus established that the IgG3-IgG1 complexes are the main components of increased intrathecal IgG in MS CSF. Further, we have established that these elevated IgG3-IgG1 complexes can also be detected in sera of MS patients. IgG3-IgG1 complexes from CSF and serum were purified from patients with PPMS and RRMS (via 2% polyethylene glycol (PEG) 8000 immune complex precipitation and collecting the flow-through after Protein A and Protein G magnetic bead binding to both CSF and sera), and the complexes were dissociated with treatments such as low pH acid treatment (Glycine-HCl, 0.1 M, pH 2.5), high pH buffer (Borate buffer, 0.1 M, pH 10.8) acid treatment plus heating/boiling, and 9 M urea. We found that using our new methods, that total protein, total IgG (heavy and light chains), and IgG subclasses (IgG1 and IgG3) levels are elevated in MS serum and CSF and can be used as the basis for assays to detect MS using sera from patients. We also found that the oligoclonal bands in MS are glycosylated IgG3, IgG1, and albumin complexes, and O-linked glycosylated (N-acetyl glucosamine) IgG3 heavy chain contributes to the increased levels of IgG in MS CSF, and correlates with MS disease progression.

However, current methodologies fail to distinguish IgG3-IgG1 and necessitates development of new methods for their detection in clinical assays of patients.

Additionally, we have developed procedures to detect OCB in paired CSF and serum by SDS-PAGE Western blots probed with either monoclonal mouse anti-human IgG3 or IgG1 antibodies, by calculating the increased ratio of band intensity of the IgG heavy chain 65 kD to the 51 kD in CSF compared to that in paired serum. These two antibodies react to both IgG3 heavy chain (65 kD) and IgG1 heavy chain (51 kD) in Western blots. So the increased ratio of the band intensity of 65 kD/51 kD in CSF compared to the paired serum corresponds to the OCB observed by current clinical lab procedure using isoelectric focusing immune blots.

The instant invention is also directed to new methods to distinguish between the different clinical phenotypes of MS [primary-progressive MS (PPMS), secondary-progressive MS (SPMS), and relapsing-remitting MS (RRMS)] which can be differentiated by, for example, SDS-PAGE Western blots of CSF and sera probed with anti-albumin antibody. We have found that the levels of IgG3-IgG1 complexes and/or the level of albumin in the serum or CSF correlates with disease progression. We show that significant amounts of IgG3-IgG1 complexes are present from MS serum and CSF flow-through after Protein A and Protein G bead binding, and these complexes appear in different amounts depending on the clinical phenotype of MS.

The efficacy of the present invention is evidenced by a number of studies. First, sera of patients with MS show elevated levels of total protein. We noticed that long-term storage (from a few days to a few months) of MS sera, but not the healthy control (HC) sera at 4° C. induces protein precipitates, suggesting the presence of high amounts of protein in MS sera. We measured total protein concentration in MS sera using BCA total protein assay and discovered that there are significantly elevated levels of total protein in MS sera compared with inflammatory controls (IC), non-inflammatory CNS disorders (NIC), and healthy controls (HC). Furthermore, after treatment of sera with 0.1 M Borate buffer pH 10.8 for 30 min at 95° C., higher levels of protein were detected in MS sera compared to controls by BCA measurement. The higher protein level in MS sera were further confirmed by Native polyacrylamide gel electrophoresis (Native PAGE) Coomassie stain of MS sera and low pH (0.2 M Glycine, pH 2.5) treated MS sera.

We also detected elevated levels of total IgG, IgG1, and IgG3 subclasses in sera of patients with MS. To analyze the unique features of IgG antibodies in sera of MS patients, we performed native PAGE followed by Coomassie staining and immunoblotting in sera of MS, IC, and HC. We demonstrated the presence of large amounts of high molecular weight (MW) IgG (greater than 230 kD) in MS sera but not in IC and HC sera when probed with anti-human IgG (H+L)-HRP. Band intensity analysis shows that MS patients on average contain 2.5-fold higher amounts of IgG than inflammatory control patients, and 4-fold more IgG than healthy controls, indicating that the increase is contributed by IgG complexes. We further performed SDS-PAGE Western blots of various preparations of sera from MS and controls, including total sera, Protein A and Protein G flow-through (FT), and probed with anti-human IgG (H+L)-HRP, rabbit/mouse anti-human IgG1 and IgG3 antibodies followed by corresponding HRP-secondary antibodies. We showed that the significantly elevated levels of IgG1 and IgG3 are present in both Protein A FT (A-FT) and Protein G-FT (G-FT), and the band intensity ratio of IgG1 (A-FT/total serum) is greater than 1 in MS, but the band intensity of IgG1 (A-FT/total serum) is less than 1 in controls. Furthermore, greater amounts of IgG1 and IgG3 are released in both low pH (0.2 M Glycine, pH 2.2) and high pH (0.1 M borate, pH 10.8) treated MS sera with heating at 95° C. or no heat. Elevated levels of total IgG was further demonstrated by ELISA of MS sera of Protein A (A-FT) and Protein G flow-through (G-FT) probed with anti-human IgG (H+L)-HRP.

We also demonstrated that the increased levels of protein in MS sera are contributed by highly elevated glycosylated IgG3, IgG1, and albumin which form immune complexes with blocked Fc region. We have performed multiple immunno-assays to demonstrate the presence of IgG immune complexes in MS, as explained below in points (a)-(h).

Point (a), significantly elevated levels of total protein, IgG1, IgG3, and albumin in MS sera can only be detected after treatment with both low (0.2 M Glycine, pH 2.2) or high pH (0.1 M borate, pH 10.8) buffer, suggesting the presence of immune complexes.

Point (b), high molecular weight IgG (150-250 kD) are only detected in native PAGE WB with anti IgG (H+L) antibody (not by anti-Fc antibody) in total sera, suggesting that the immune complexes contain blocked Fc region, and further support the presence of immune complexes.

Point (c), PEG-8000, a known agent to purify immune complexes (Ohlson, 1984) can precipitate more than 3-fold more of IgG1, IgG3, and total protein in MS sera than healthy controls.

Point (d), the higher levels of total IgG, IgG1 and IgG3 can only be detected by denatured assay (SDS-PAGE Western Blot), but not by immune assays with non-denatured proteins such as ELISA and slot blots (dot blots) (native un-denatured protein) which showed lower levels of total IgG, IgG1 and IgG3 compared to inflammatory controls.

Point (e), we found significantly elevated galactosylated IgG (detected by Ricinus communis Agglutin I (RCA) in Western Blot) in MS sera.

Point (f), SDS-PAGE Western Blots show that Protein A purification depleted most of the IgG heavy (H) and light (L) chains in MS CSF and serum, and most of the glycosylated IgG detected by 3 common biotinylated lectin probes (Griffonia simplicfolia Lectin II for N-acetylglucosamine; Sambucus nigra Lectin for sialic acid; Ricinus communis agglutinin Agglutin I for galactose) from MS CSF and sera, suggesting that the immune complexes do not bind Protein A well. In addition, SDS-PAGE Western blot ratio of band intensity of IgG1 (A-FT/total) is greater in MS than controls, indicating that after addition of Tween²⁰, a surface tension lowering agent, the immune complexes were broken releasing higher amounts of IgG1.

Point (g), MS sera and Protein G-FT and Protein A-FT produce complement independent cytotoxicity on mouse primary neuronal cells, suggesting that the IgG complexes are cytotoxic, implying that the increased IgG3-IgG1 immune complexes in the brain of MS patients may produce damage in similar way.

Point (h), in PPMS CSF, higher levels of IgG3-IgG1 immune complexes are present, and detection of IgG3 is blocked on first Western Blots. Higher levels of IgG3 heavy chain can only be revealed after stripping Western blots with stripping buffer (100 mM 2-Mercaptoethanol, 2% SDS, 62.5 mM Tris-HCl pH 6.8, 0.1 M Glycine pH 2.2, SDS, etc.), indicating that the IgG complexes can be completely dissociated in the presence of surface tension lowering agent.

We also found that O-linked glycosylated IgG3 heavy chain contributes to the increased levels of IgG in MS CSF, and the oligoclonal bands in MS are IgG3-IgG1-albumin complexes which correlate with MS disease progression. There is no correlation between levels of CSF IgG concentration and numbers of oligoclonal bands (R=0.1) based on the clinical data. The clinical IgG concentration data was obtained using the Siemens BN™ II nephelometry system detecting human IgG with anti-Fc monoclonal antibody. Although it is well established that increased intrathecal IgG is a consistent and characteristic feature of MS, lower levels of IgG and albumin concentration were detected in MS CSF compared to inflammatory controls (based on the clinical data). This conflicting results indicate the presence of IgG complexes with blocked Fc region which are not detected by nephelometry using anti-Fc Ab. Further evidence of the presence of IgG immune complexes in the CSF come from the following evidence:

Point (a). Using clinical data, there is a correlation between albumin and IgG in MS CSF (r=0.44, p=0.001, n=53), but not in the serum (r=−0.008, p=0.6, n=51).

Point (b). Lower levels of total IgG and IgG3 were detected in MS CSF by Slot blots (non-denatured protein), but higher levels of IgG3 only (not total IgG, nor IgG1) are found in MS CSF.

Accordingly, we have developed novel lab procedures to assess the level of severity of MS or disease progression in a patient diagnosed with MS, and differentiate MS from other inflammatory CNS diseases. PPMS, secondary progressive MS (SPMS), and RRMS can be differentiated by SDS-PAGE Western blots of CSF and sera samples of total, Protein G, and Protein A purified IgG, and Protein A and G flow through probed with anti-human IgG (H+L), anti-human IgG1 and anti-human IgG3 antibodies. Absence of IgG light chains in SPMS, and lower levels of albumin were found in SPMS and PPMS. Furthermore, MS and inflammatory controls (IC) can be differentiated by Western blot of sera of A-FT which show that higher levels of albumin (ALB) in IC but less in MS. Additionally, Western Blot of MS and IC CSF with IgG3 probe showed that MS CSF contained much lower levels of IgG3, but the higher levels can be revealed only after stripping the membrane and reprobe with anti-IgG3 antibody, further confirming the presence of IgG3 complexes in MS CSF.

Therefore, in one embodiment, the present invention is directed to a method to treat a patient having multiple sclerosis. The method includes a step of determining whether the patient has an elevated level of human IgG in a tissue sample, such as, for example, in the blood, relative to an appropriate control, in order to determine whether the patient has MS. Alternatively, this method comprises selecting a patient having, or at risk of having MS, by determining whether the patient has an elevated level of human IgG relative to an appropriate control. The methods further comprise treating a patient identified by the methods disclosed herein with a pharmaceutical compound that is effective for treating MS. This method also includes the step of diagnosing a patient with MS or with a likelihood of having MS, or selecting a patient for further testing for MS should the patient show elevated level of human IgG relative to an appropriate control.

Therefore, in one embodiment, the method comprises a step of obtaining a sample from a patient. Obtaining a sample of the patient's tissue may be done by any methods known in the art. In one embodiment, the sample is a serum sample, obtained from a routine blood draw by methods known in the art. In another embodiment, the sample is cerebrospinal fluid obtained by methods known in the art, e.g., by lumbar puncture. Lumbar puncture is commonly carried out under sterile conditions by inserting a needle into the subarachnoid space, usually between the third and fourth lumbar vertebrae. CSF is extracted through the needle. As used herein a sample generally can be from any organs in human and can further include, but is not limited to, peripheral blood, plasma, urine, saliva, gastric secretion, cerebrospinal fluid (CSF), feces, bone marrow specimens, primary tumors, metastatic tissue, embedded tissue sections, frozen tissue sections, cell preparations, cytological preparations, exfoliate samples (e.g., sputum), fine needle aspirations, amino cells, fresh tissue, dry tissue, and cultured cells or tissue. It is further contemplated that the biological sample of this invention can also be whole cells or cell organelles (e.g., nuclei). The amount of sample to obtain can be determined by one of skill in the art, and generally is minimized as much as possible. For example, the amount of a sample of blood to obtain may be as little as 0.5 ml, and CSF may be less than 1 ml of sample.

Immunoglobulin G (IgG) a protein composed of four peptide chains—two identical heavy chains and two identical light chains arranged in a Y-shape typical of antibody monomers. IgG is the most common type of antibody found in the circulation and represent about 75% of circulating antibody in humans. IgG molecules are created and released by plasma B cells.

As discussed elsewhere herein, it is the inventor's insight, without being bound by theory, that IgG1 and IgG3 become complexed in MS. It is believed that the co-presence of IgG1 and IgG3 antibodies may play a significant role in MS disease activity, including the hypothesis that the IgG3-IgG1 complexes are a strong candidate as a disease marker in progressive MS. Specifically, the inventor has developed evidence tending to show that MS sera have increased levels of protein which are contributed to by highly elevated glycosylated IgG3, and IgG1, and albumin which form immune complexes. These immune complexes appear to have a blocked Fc region, which hinders the ability to detect the presence of IgG when non-dissociated samples are probed with reagents that are specific for detecting an Fc region. The presence of immune complexes is evidenced by the fact that significantly elevated levels can be detected after dissociation of the complexes (by, for example, 0.2 M glycine pH 2.2 or 0.1 M borate, pH 10.8 buffer).

These complexes may be pathogenic, as evidenced by work by others showing that purified and recombinant intrathecal antibodies in MS targeted CNS cells; CSF of MS patients induced axonal damage and apoptosis in culture; anti-myelin antibodies of MS serum produce demyelination, serum antibodies of MS patients target microvessels in brain tissues, and sera from RRMS and SPMS disrupt the blood-brain barrier. These data, combined with a recent demonstration of B cells in the periphery participating in OCB production in MS CSF, further indicate that antibodies in both serum and CSF of MS may be pathogenic. The inventors, without being bound by theory, have found that the apparent complexation of IgG1 and IgG3 interferes with the detection and assay of IgG1 and IgG3 in both serum and CSF. In fact, it is commonly understood that enhanced levels of IgG1 and IgG3 (i.e., the oligoclonal bands or OCB) in MS can only be detected in CSF, as in MS enhanced levels of IgG1 and IgG3 are not observable in serum.

Immune complexes are formed by the interaction of antibodies with specific antigens. Complement activation is the most important biological function of IgG. IgG immune complexes can activate all three pathways of the complement system, resulting in the generation of C3 and C5 cleavage products, which can activate a panel of different complement receptors on innate and adaptive immune cells. A very significant correlation between plasma C3d levels and circulating immune complexes is observed in rheumatoid arthritis. Activation of the classical pathway takes place when C1q recognizes IgG molecules that form immune complexes with their cognate antigens. Similarly, unspecific aggregation of such antibodies and activation of C1q have been described in cryoglobulinemia. In the case of MS, IgG3 may act as self-antigen, forming immune complexes with the abundant autoantibody IgG1. Subsequently, the IgG3-IgG1 complexes may activate the classical pathway by markedly increasing the binding affinity of C1q, leading to tissue damage caused by significant and dynamic systemic activation and up regulation of complement.

It is the observation of the inventor that the immune complexes that are formed will “mask” sites such as the Fc region of IgG, which consequently lead to interference with common methods of detection of enhanced amount of IgG or OCB.

Therefore, the methods of the instant invention include the step of dissociating and/or denaturing any immune complexes that are present in the patient's sample. Dissociation generally refers to the separation of more than one unit into individual units, which may or may not be capable of re-associating. Denaturation generally refers to a loss of tertiary and/or secondary structure (or quaternary structure) leading to separation into, for example, monomers. Dissociation and/or denaturation of the immune complexes may be carried out by any methods known in the art. For example, protein-protein complexes which comprise complexes of IgG1, IgG3, and combinations thereof may be dissociated by, for example, changes in pH, such as an acidic treatment, a basic treatment; changes in temperature such as a heat treatment, detergents; agitation; chaotropic agents, or combinations thereof. Suitable amounts of each and concentration of each to effect dissociation and/or denaturation can be determined by one of skill in the art. The extent of dissociation and/or denaturation may be measured by one of skill in the art by, for example, Native PAGE Western blots probed with anti-human IgG (H+L) and Coomassie stain showing an increase and/or decrease in high molecular weight bands indicating increase and/or decrease of detectable or measurable IgG and total protein.

In one embodiment, dissociation may be effected by addition of 0.1 M Borate buffer at a pH of above 10 at 1:1 to 1:30 or higher.

In this embodiment, or in addition to this embodiment, optionally, the method may further include contacting the sample with at least one reagent which is capable of specific binding to immunoglobulins. In some embodiments, the sample is diluted prior to contacting the sample with any reagents. For example, the sample can be diluted into an isotonic buffer such as PBS (phosphate buffered saline), for example, in a dilution of about 1:10, 1:100, and so on, as determined by one of skill in the art for maximizing convenience, accuracy, reproducibility of the results, and minimization of expense in purchasing reagents. In one embodiment, this reagent comprises Protein A, Protein G, Protein A/G and/or Protein L. In another embodiment, the reagent comprises anti-human IgG-Fc specific. The sample may be contacted with a single reagent, or multiple reagents may be used together or sequentially, for example, the method may include contacting the sample with Protein A, followed by contacting the sample's non-bound portion from Protein A, with anti-human IgG1 antibody or anti-human IgG (H+L). In addition, the method may include contacting the sample with PEG 8000 to bring to final concentration of 2% PEG and precipitate the IgG complexes followed by measurements of SDS PAGE Coomassie stain to detect increased levels of total protein.

It is conventionally believed in the art that reagents which are capable of specific binding to immunoglobulins such as Protein A, binds to IgG generally, and also binds to IgG1 with lesser binding to IgG3, whereas Protein G binds to IgG generally, as well as to IgG1 and IgG3. Therefore treatment of a sample with at least one of Protein A, Protein G and/or Protein A/G would be expected, by one of skill in the art, to bind to IgG, IgG1 and/or IgG3 in the sample. However, surprisingly, the inventors found that rather than being bound by the Protein A, Protein G, and/or Protein A/G, the IgG in the sample was not bound efficiently. Instead, the IgG (IgG1 and/or IgG3) appeared in the nonbound fraction upon separation of bound and unbound fractions, e.g., IgG (IgG1 and/or IgG3) was in the “flow through” fraction or nonbound fraction. Without being bound by theory, it is believed that the lack of expected binding can be attributed to complexation of IgG which obscures and/or masks binding sites on the IgG (IgG1 and/or IgG3) such as the Fc portion of the IgG.

Therefore, the inventors have found that the IgG (IgG1 and/or IgG3) complexes observed in MS samples are enriched relative to other proteins and/or antibodies upon treatment with a reagent comprising PEG 8000, Protein A, Protein G, or Protein A/G. Protein A, Protein G, or Protein A/G reagents are well known in the art and available from a number of suppliers.

The type of reagent to use can be selected by the skilled person in accordance with the type of assay used. For example, ThermoFisher Scientific (Waltham, Mass.) makes available surface coated microplates coated with Protein A, Protein G, Protein A/G which is suitable for an ELISA format. For other formats, such as Western blotting, a resin or bead coated with a reagent which binds immunoglobulins may be used. For example, ThermoFisher Scientific makes available DYNABEADS Protein A, Protein G, or Protein A/G magnetic beads for immunoprecipitation.

The step of contacting the sample with the reagent can be accomplished by any means known in the art. Optionally, the step comprising treatment with the reagent which can include Protein A, Protein G and/or Protein A/G can include a dissociation step. For example, the sample and Protein A, Protein G, and/or Protein A/G may be incubated in a buffer which is capable of dissociating immune complexes, such as, wherein the buffer comprises PBS-Tween 20 (0.05%).

The nonbound fraction of the sample may be collected by any means known in the art, suitable to the reagent, such as, for example, removal of the supernatant from coated plates or removal of the supernatant from the resin.

In another embodiment to collecting a nonbound fraction of the sample from a reagent that binds immunoglobulin as outlined above, the method also optionally includes the step to selectively precipitate protein complexes, or preferably, selectively precipitate immune complexes. Any method known in the art to precipitate immune complexes is suitable and exact conditions can be determined by the skilled person. For example, the sample may be contacted with a polyethylene glycol to selectively precipitate immune complexes of IgG1 and IgG3. and collecting the precipitate for use as the sample in the further method steps. In one embodiment, the polyethylene glycol is PEG-8000 and the concentration to use to selectively precipitate immune complexes is at 2% final PEG concentration. The precipitated immune complexes may be resolubilized and optionally, dissociated, and used as the sample in the further steps in the method.

The method also includes the step of detecting and/or quantitating the human IgG in the sample with a reagent that binds specifically to a human IgG. As stated above, the sample can include a sample which has been contacted with Protein A, Protein G, Protein A/G, or L and the nonbound portion thereof can be used as the sample. Alternatively, the sample may be the resolubilized, precipitated sample after selective precipitation for protein complexes, such as immune complexes. This step optionally includes a step wherein the amount of human IgG is quantitated.

A reagent that binds specifically to a human IgG includes a reagent that binds to IgG heavy and light chain generally, or to specific subtypes or classes of IgG, such as IgG1 and/or IgG3. In one embodiment, the reagent that binds specifically to a human IgG includes reagents such as antibodies, fragments thereof, or a lectin. The reagents may further include a moiety which assists in visualization and/or detection, as is known in the art, for example, a biotinylated lectin. In some embodiments, the reagent that binds specifically to a human IgG includes an antibody or fragment thereof. The term “antibody” is meant to also encompass immunologically effective fragments of antibodies. As far as fragments of an antibody are concerned, it is preferred that the fragments retain an antigen-binding domain and an Fc-domain. The antibody or fragment thereof preferably binds selectively to at least one of the following: whole human IgG (heavy chain and light chain), a human IgG heavy chain, a human IgG light chain, human IgG subclass IgG1, human IgG subclass IgG3, human Fc, and a human glycosylated IgG.

The format in which to perform this detection can be determined by one of skill in the art. In one embodiment, the format is an Enzyme-linked immunosorbent assay (ELISA). Performing an ELISA requires at least one capture step, at least one detection antibody, and/or at least one enzyme-linked or fluorescent labeled secondary antibody. For example, assaying the amount of IgG by ELISA may require a capture step to bind the IgG to the microtiter plate, which can include use of an antibody as a capture reagent. In this embodiment, a capture antibody is immobilized on a solid support such as a polystyrene microtiter plate. The sample is then added and allowed to complex with the bound antibody. Unbound fraction of the sample can be removed with a wash. A detection antibody, e.g., a monoclonal or polyclonal antibody or lectins that binds selectively to whole human IgG (heavy chain and light chain), a human IgG heavy chain, a human IgG light chain, human IgG subclass IgG1, human IgG subclass IgG3, human Fc, and a human glycosylated IgG, may be added and is allowed to bind to captured IgG. The detection antibody is linked to an enzyme, either directly or indirectly, e.g., through a secondary antibody that specifically recognizes the detection antibody. Typically between each step, the plate, with bound IgG, is washed with a wash buffer, e.g., a mild detergent solution. Typical ELISA protocols also include one or more blocking steps, which involve use of a non-specifically-binding protein such as bovine serum albumin to block unwanted non-specific binding of protein reagents to the plate. After a final wash step, the plate is developed by addition of an appropriate enzyme substrate, to produce a visible signal, which indicates the quantity of IgG in the sample. The substrate can be, e.g., a chromogenic substrate or a fluorogenic substrate, for example. Suitable antibodies are commercially available, from, for example, Life Technologies (Carlsbad, Calif.). ELISA methods, reagents and equipment are well-known in the art and commercially available.

In another embodiment, the format for detection and/or quantitation is a Western blot. A Western blot is well-known in the art and is a widely used analytical technique used to detect specific proteins in a sample. Gel electrophoresis can be used to separate native proteins by 3-D structure or denatured proteins by the length of the polypeptide. The proteins are then transferred to a membrane (typically nitrocellulose or PVDF), where they are stained with antibodies specific to the target protein. Accordingly the Western blot includes a step of carrying out denaturing polyacrylamide gel electrophoresis (PAGE), such as, for example, SDS PAGE, on the sample, followed by the step of detecting and/or quantitating the human IgG in the gel with a reagent that binds specifically to a human IgG, followed by chemiluminescent detection. As an example, the reagent can be HRP-anti-human IgG (H+L) followed by an ABTS color reaction.

Alternatively, a dot blot (slot blot) may be used, in which the sample is not first separated by electrophoresis, and instead, the sample is applied directly on a membrane (nitrocellulose or PVDF, for example) as a dot and then followed by detection by an antibody as described hereinabove.

In order to determine whether the amount of IgG is elevated, the amount of IgG is compared to an appropriate control. An appropriate control may be determined by one of skill in the art. An appropriate control will include a sample from a healthy donor, or an average of samples from a healthy control. Alternatively, an appropriate control may include a sample from a patient with an inflammatory CNS disease (inflammatory control) or an average of samples from inflammatory control. Controls comprising healthy donors include, for example, pooled human sera or standard pooled purified human IgG from human serum. Use of an inflammatory control sample or average of inflammatory control samples may be appropriate in some circumstances when it is difficult to differentiate inflammatory CNS disorders from MS even by detection of OCB, because OCB can be found in both MS and inflammatory CNS disorders. Alternatively, an appropriate control may include a sample from a patient with a non-inflammatory CNS condition or an average of samples with non-inflammatory CNS condition. Use of a non-inflammatory CNS condition control sample or average thereof may be appropriate in some circumstances, such as when they both show similar clinical presentations In some embodiments, an MS patient will contain, on average, approximately 2.5-fold higher amounts of IgG than inflammatory control patients, and 4-fold more IgG than healthy controls.

The method may further include the step of administering a pharmaceutical compound effective for treating MS to the patient having an elevated level of IgG. The most common initial course of the disease is the relapsing-remitting subtype (RRMS), which is characterized by unpredictable attacks (relapses) followed by periods of relative remission with no new signs of disease activity. After some years, many of the people who have this subtype begin to experience neurologic decline without acute relapses. When this happens it is called secondary progressive multiple sclerosis (SPMS). Other, less common, courses of the disease are the primary progressive (PPMS) (decline from the beginning without attacks) and the progressive-relapsing (steady neurologic decline and superimposed attacks). Different therapies are used for patients experiencing acute attacks, for patients who have the relapsing-remitting subtype, for patients who have the progressive subtypes, for patients without a diagnosis of MS who have a demyelinating event, and for managing the various consequences of MS, as is known in the art.

For example, an approved MS disease-modifying treatment may be used as appropriate, such as, for example, interferon beta-1a (Avonex, Rebif, CinnoVex, ReciGen, Plegridy), interferon beta-1b (Betaseron), glatiramer acetate (Copaxone), mitoxantrone (Novantrone), natalizumab (Tysabri), fingolimod (Gilenya), teriflunomide (Aubagio),dimethyl fumarate (BG12, Tecfidera) and alemtuzumab (Campath, Lemtrada), or combinations thereof. Other drugs are under investigation such as laquinimod, PEGylated versions of interferon-β-1a, and monoclonal antibodies with the same target as natalizumab such as daclizumab and CD20 monoclonal antibodies such as rituximab, ocrelizumab and ofatumumab. These therapies are used in accordance with methods known in the art, and include administering therapeutic amounts of one or more of these treatments as a single dose or as a course of treatment as recommended by the manufacturer.

The methods of the invention may be used as an adjunct to conventional methods to diagnose MS, as are known in the art. For example, the methods of the invention may be used for more general screening of patients. Where patients tested positive for MS or clinical subtypes of MS by the methods of the invention, these positive patients may be selected for further testing for MS by alternative methods for diagnosing MS, such as those known in the art. For example, further or additional testing for MS may be carried out by methods such the demonstration of MS-typical CNS lesions disseminated in space and time by MRI, or a combination of clinical and MRI findings; alternatively, testing for MS may be carried out by presence of OCB. The qualitative measurement of elevated IgG in the CSF of MS patients (this method is currently the only laboratory biomarker included in MS diagnostic criteria),via Isoelectric focusing (IEF) immune blots, for example,) is the best lab method for detection of oligoclonal bands (OCBs). Alternatively, patients which test positive for MS by other methods known in the art may then be tested with the methods of the instant invention.

In another embodiment, the present invention includes a method to treat a patient having multiple sclerosis by determining the level of at least one protein relative to an appropriate control. The method includes a step of determining whether the patient has an elevated level of at least one protein relative to an appropriate control, in order to determine whether the patient has MS. Alternatively, this method comprises selecting a patient having, or at risk of having, MS, by determining whether the patient has an elevated level of at least one protein relative to an appropriate control. The methods further comprise treating a patient identified by the methods disclosed herein with a pharmaceutical compound that is effective for treating MS. This method also includes the step of diagnosing a patient with MS or with a likelihood of having MS, or selecting a patient for further testing for MS should the patient show elevated level of at least one protein relative to an appropriate control.

The method includes a step of determining whether the patient has an elevated level of at least one protein relative to an appropriate control, in order to determine whether the patient has MS. Alternatively, this method comprises selecting a patient having, or at risk of having, MS, by determining whether the patient has an elevated level of at least one protein relative to an appropriate control. The methods further comprise treating a patient identified by the methods disclosed herein with a pharmaceutical compound that is effective for treating MS. This method also includes the step of diagnosing a patient with MS or with a likelihood of having MS, or selecting a patient for further testing for MS should the patient show elevated level of at least one protein relative to an appropriate control.

In this embodiment, the method includes the steps of obtaining a sample from the patient by methods of the invention, and a method to dissociate immune complexes between IgG1 and IgG3.

The method further includes measuring the amount of at least one protein. In this embodiment, the term “protein” comprises any protein that is elevated in CSF and/or serum of a patient having MS. Such proteins include, without limitation, total protein, albumin, IgG3 or IgG1, glycosylated IgG, glycosylated IgG3, glycosylated IgG1, and/or O-linked glycosylated IgG3. Methods to measure protein concentrations, including the specific proteins listed herein, are known in the art and an appropriate method may be determined by one of skill in the art. For example, a reagent capable of measuring total protein includes a BCA reagent which is a colorimetric assay, and SDS PAGE Coomassie stain. Measurement of albumin, IgG3 or IgG1, glycosylated IgG, glycosylated IgG3, glycosylated IgG1, and/or O-linked glycosylated IgG3 may be carried out by a reagent that specifically binds to the protein to measure. For example, without limitation, an antibody specific to human albumin, IgG3 or IgG1, glycosylated IgG, glycosylated IgG3, glycosylated IgG1, and/or O-linked glycosylated IgG3 may be used, such as an antibody specific to the protein of interest, a lectin, or any other reagent capable of binding to the protein of interest.

The amount of protein in the sample is determined and the presence of an elevated level of the protein is determined by comparison to a control, as defined elsewhere herein.

The method also optionally includes the step of dissociating and/or denaturing any immune complexes and/or any protein complexes that are present in the patient's sample, as disclosed elsewhere herein. The method may also further comprise contacting the sample with at least one reagent which is capable of specific binding to immunoglobulins, as disclosed elsewhere herein, such as Protein A, Protein G, Protein A/G and/or Protein L, and using the unbound portion of the sample; alternatively, a selective precipitation method as disclosed elsewhere herein may be used to selectively enrich for a protein complexes which is elevated in MS.

There are different clinical presentations of MS throughout the course of the disease, and therapies/treatments may be adjusted in accordance with the clinical presentation of MS in the patient. The most common initial course of the disease is the RRMS subtype, which is characterized by unpredictable attacks (relapses) followed by periods of relative remission with no new signs of disease activity. After some years, many of the people who have this subtype begin to experience neurologic decline without acute relapses. When this happens it is called secondary progressive multiple sclerosis. Other, less common, courses of the disease are the PPMS (decline from the beginning without attacks) and the progressive-relapsing (steady neurologic decline and superimposed attacks). The present invention also provides methods to distinguish which type of MS a patient may have, and/or diagnose a patient with PPMS and/or RRMS.

Accordingly, this embodiment includes a method to diagnose a patient with primary-progressive MS (PPMS) and/or relapsed-remitting MS (RRMS). The methods further comprise treating a patient identified by the methods disclosed herein with a pharmaceutical compound that is effective for treating MS. This method also includes the step of diagnosing a patient with MS or with a likelihood of having MS, or selecting a patient for further testing for MS should the patient be diagnosed with PPMS by methods of the invention.

This method can include the steps as described hereinabove, namely, obtaining a sample from the patient, treating the sample to dissociate immune complexes of IgG1 and IgG3, contacting the sample from the patient with a reagent which binds specifically to a human IgG and/or a human albumin; and comparing the amount of IgG, albumin, or both IgG and albumin, in the sample with an appropriate control.

The appropriate control can vary. In one embodiment, where the method is directed to determining whether a patient has PPMS, an appropriate control can include a patient or patient pool having relapsing-remitting MS (RRMS). Where the patient or patient pool has RRMS, then if the comparison with the sample shows a reduced level of albumin and/or an increased level of IgG as compared to the control, then the patient may have PPMS. Alternatively, if the sample shows a similar level of albumin and/or IgG as the patient or patient pool having RRMS, then the patient may have RRMS. PPMS (and SPMS) patients have been found to have increased IgG3-IgG1 complexes compared to RRMS and do not bind well to Protein A or G, therefore produce less IgG when purified, but leave most in the flow through. So for Protein A and G purified IgG: RRMS>SPMS>PPMS; for IgG3-IgG1 complexes: PPMS>SPMS>RRMS.

Alternatively, for diagnosis of PPMS, the control can be a patient or patient pool having primary-progressive MS (PPMS). Where the patient or patient pool has PPMS, then if the comparison shows an equivalent level of albumin and/or an equivalent level of IgG as compared to the control, then the patient may have PPMS.

In another embodiment, for diagnosis of RRMS, the control can be a patient or patient pool having primary-progressive MS. Where the patient or patient pool has PPMS, then if the comparison shows enhanced levels of albumin and/or reduced levels of IgG, then the patient may have RRMS.

The method also optionally includes the step of dissociating and/or denaturing any immune complexes and/or any protein complexes that are present in the patient's sample, as disclosed elsewhere herein. The method may also further comprise contacting the sample with at least one reagent which is capable of specific binding to immunoglobulins, as disclosed elsewhere herein, such as Protein A, Protein G, Protein A/G and/Protein L, or anti-human IgG Fc antibody, and using the unbound portion of the sample; alternatively, a selective precipitation method as disclosed elsewhere herein may be used to selectively enrich for a protein or protein complexes which is elevated in MS.

The method may further include the step of administering a pharmaceutical compound effective for treating MS to the patient, which can be determined by one of skill in the art depending on whether the patient is diagnosed with RRMS or PPMS, as disclosed elsewhere herein.

In another embodiment, the present invention includes nondenaturing methods to determine whether a patient has elevated immune complexes of IgG (such as, complexes of IgG1 and/or IgG3), and/or reduced levels of detectable/measurable IgG (such as, total IgG, IgG1 or IgG3) to determine whether a patient has MS. The method includes a step of determining whether the patient has elevated levels of immune complexes of IgG (such as, complexes of IgG1 and/or IgG3), and/or reduced levels of detectable/measurable IgG (such as, total IgG, IgG1 or IgG3) relative to an appropriate control, in order to determine whether the patient has MS. Alternatively, this method comprises selecting a patient having, or at risk of having, MS, by determining whether the patient has elevated levels of immune complexes of IgG (such as, complexes of IgG1 and/or IgG3), and/or reduced levels of detectable/measurable IgG (such as, IgG1 or IgG3) relative to an appropriate control. The methods further comprise treating a patient identified by the methods disclosed herein with a pharmaceutical compound that is effective for treating MS. This method also includes the step of diagnosing a patient with MS or with a likelihood of having MS, or selecting a patient for further testing for MS should the patient show elevated levels of immune complexes of IgG (such as, complexes of IgG1 and/or IgG3), and/or reduced levels of detectable/measurable IgG (such as, IgG1 or IgG3) relative to an appropriate control.

The method includes the steps of obtaining a sample from a patient and determining whether the patient has at least one of a reduced level of detectable/measurable human IgG and/or an increased level of high molecular weight IgG immune complexes (such as, for example, complexes of IgG1 and/or IgG3), when compared to appropriate controls, as discussed elsewhere herein.

In this embodiment, methods to determine whether high molecular weight immune complexes are present are utilized. Such methods are known in the art, and generally include and/or employ a method to separate proteins by molecular weight prior to analysis. For example, a common method to separate molecules by molecular weight prior to analysis is native (nondenaturing) PAGE, followed by a Western blot to detect IgG as discussed elsewhere herein. Because the immune complexes have a higher apparent molecular weight than IgG, the immune complexes will migrate on native PAGE in such a way as to indicate a higher apparent molecular weight. Comparison of the samples with an appropriate control, as selected by one of skill in the art, and disclosed in more detail elsewhere herein, can allow for determination of whether elevated levels of immune complexes and/or reduced levels of IgG are present in the sample.

In yet another embodiment, the present invention provides methods to determine the efficacy of an MS drug, or a course of treatment for MS, by the methods of the invention, as disclosed herein. In this embodiment, the elevated or reduced levels of proteins disclosed herein which are useful for diagnosing MS, selecting patients for treatment with MS, and the like, can be used as a measurable indicator of the progress or remission of the disease state (MS). The method can include obtaining samples (blood or CSF) from patients before, during and/or after drug treatment or a course of drug treatment. In this embodiment, the methods include dissociating IgG3-IgG1 immune complexes with methods described above followed by SDS PAGE Western blots, Coomassie stain and/or ELISA evaluating the reduced levels of total protein, total IgG (H+L), IgG1, IgG3 and increased levels of albumin which indicate that the patient is responsive to the therapy.

As described above, the proteins disclosed herein which are useful for determining or indicating whether a patient has MS can be used to measure the progress (or lack thereof) of MS, evaluating the most effective therapeutic regimes for the MS type, and establishing susceptibility to MS or its recurrence. Thus, the proteins disclosed herein can be useful in early diagnosis, disease prevention, drug target identification, drug response and the like.

Therefore, the methods herein directed towards determining levels of the proteins identified herein can provide a method for determining the efficacy of the drug treatment and/or the effect of the drug treatment on development of the disease in the patient. These markers may also give information on selecting a course of treatment for MS. MS is a long term chronic disease and patients may have to take medications for years. Therefore accurate diagnosis and evaluation of these drug treatments is important, especially when patients respond to a drug differently, and/or where side effects are expected from the drug treatments. Many MS drugs have significant side effects.

Thus, the present invention also includes a method to determine efficacy of an MS drug or drug candidate in a patient, which includes obtaining a sample from a patient having been treated the drug or drug candidate, by methods as disclosed herein.

For example, one may obtain samples (blood or CSF) from patients before and after drug treatment, dissociate IgG3-IgG1 immune complexes with methods described above followed by Native/SDS-PAGE Coomassie stain and Western blots, and/or ELISA evaluating the reduced levels of total protein, total IgG, IgG1 and IgG3 and increased levels of albumin which indicate that the patient is responsive to the therapy.

### EXAMPLES

The following examples are provided for illustrative purposes only and are not intended to limit the scope of the invention.

### Example 1

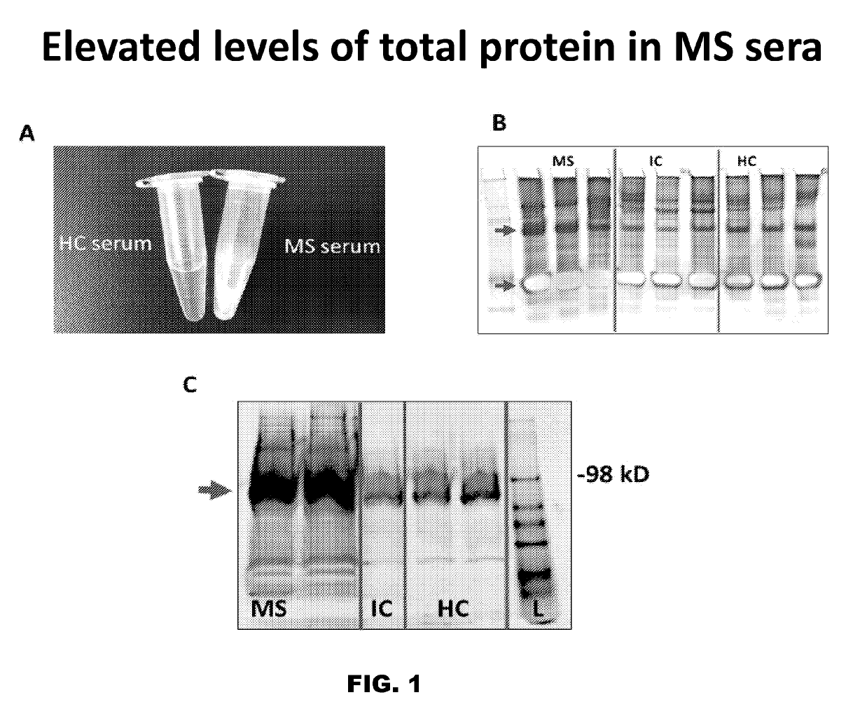

Total protein levels are higher in MS sera. FIG. 1 (a) shows that long term storage of MS sera at 4° C. induces increased protein precipitates compared to healthy control (HC). FIG. 1 (b) shows that higher levels of high molecular weight proteins were detected in Native PAGE by Coomassie blue stain for MS sera compared to control. FIG. 1 (c) shows that higher protein levels are detected in reduced and denatured PAGE stained by Coomassie blue in MS sera compared to control. Arrows indicate the proteins which shower higher levels in MS sera. IC: inflammatory CNS control; HC: healthy control.

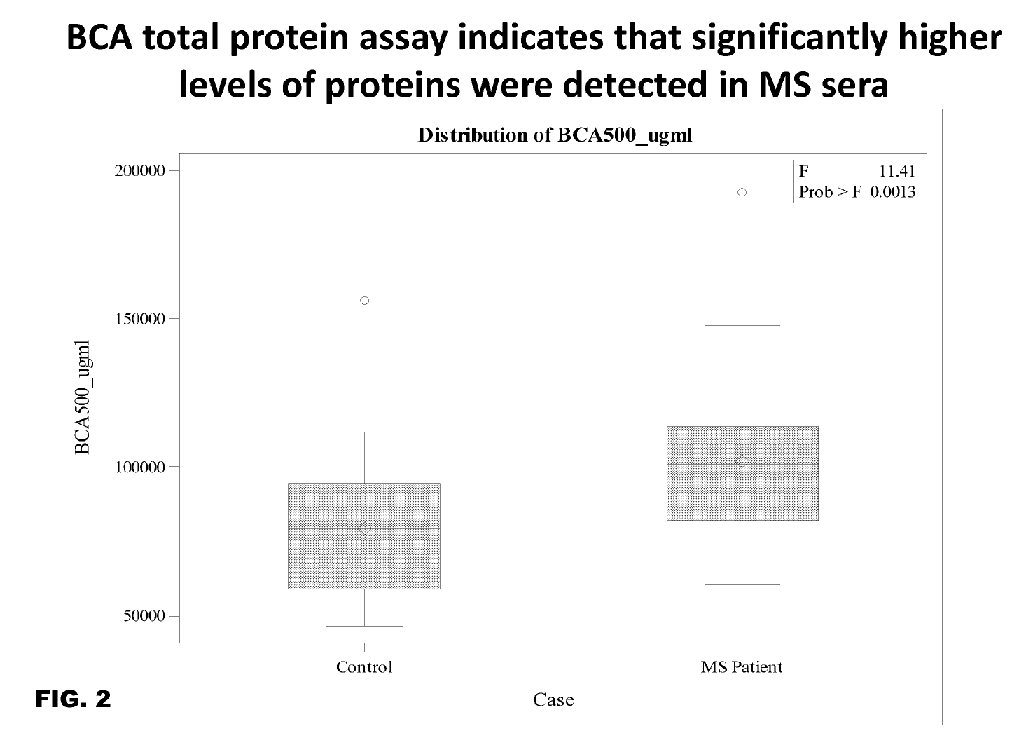

FIG. 2 shows that BCA total protein assay indicating that significantly higher levels of proteins were detected in MS sera compared to controls. 60 total samples (35 MS and 25 controls, p=0.001). Controls: 7 HC, 5 IC, 13 other neurological disorders.

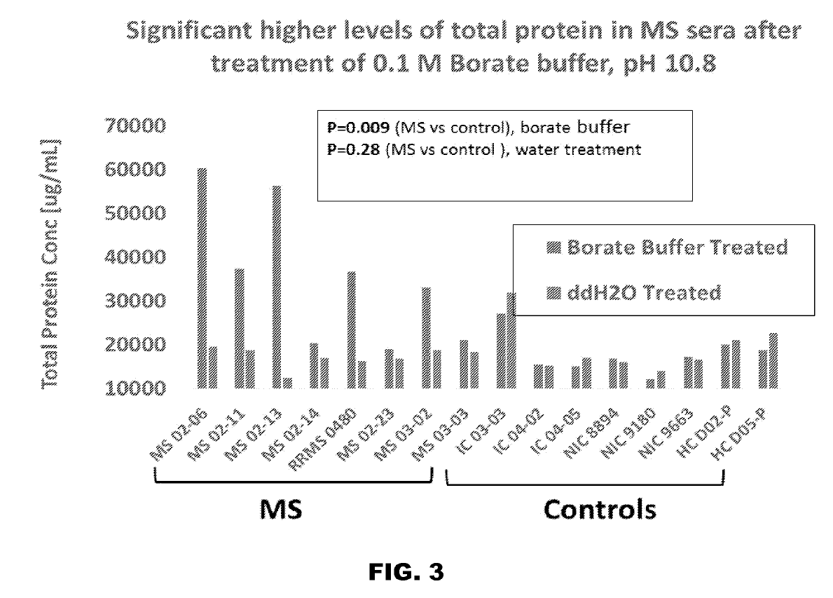

FIG. 3 shows that protein levels detected by BSA assay in MS sera are higher compared to healthy control sera after borate buffer treatment at pH 10.8 to disrupt protein complexes. Significantly elevated levels of total protein were detected in MS sera after treatment with 0.1 M Borate buffer, pH 10.8. Borate buffer was added to sera samples and heated at 95° C. for 30 min. Samples were put on ice followed by BCA total protein assay. Borate buffer treatment released significant higher amount of total protein in MS sera but not in control sera (p=0.009) [inflammatory (IC), non-inflammatory controls (NIC) and healthy controls (HC)], but no effect on total protein levels when samples were treated with water (p=0.28).

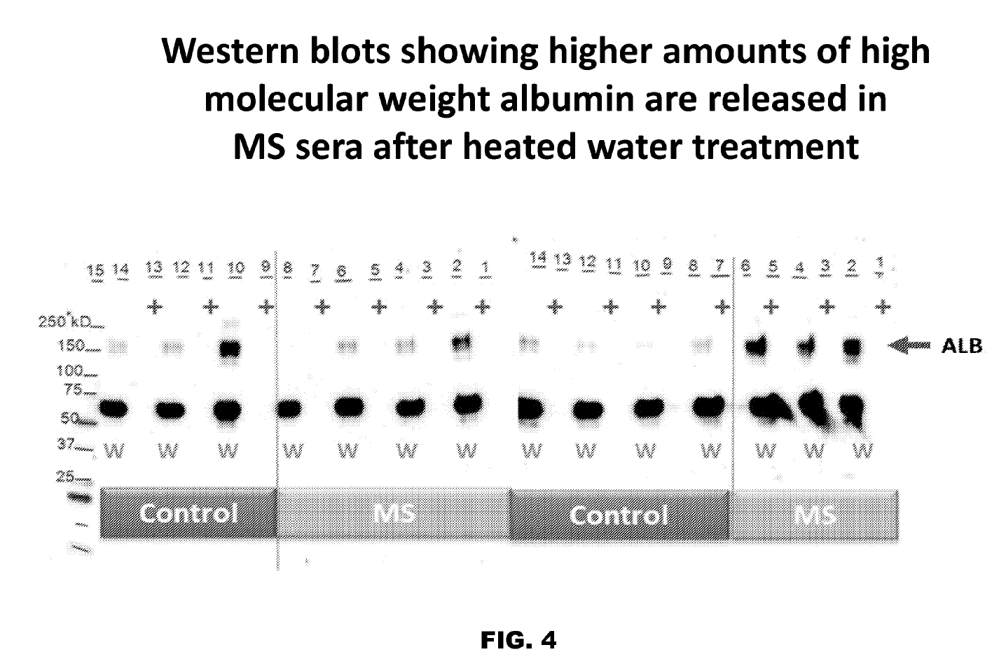

FIG. 4 shows an increase in 150 kD molecular weight band in water treated and heated sample when probed with anti-human albumin on SDS PAGE Western blots. Samples treated with water (W) and Borate buffer (+) were electrophoresed by SDS-PAGE and a Western blot of the gel was probed with anti-human albumin antibody followed by HRP-secondary antibody. Water treated MS sera showed increased levels of 150 kD band compared to controls. On average, MS sera contain at least 2.5 times higher amounts of albumin than the control sera (by band intensity analysis). The plus sign indicates samples treated with Borate buffer, and W indicated water treatment.

### Example 2

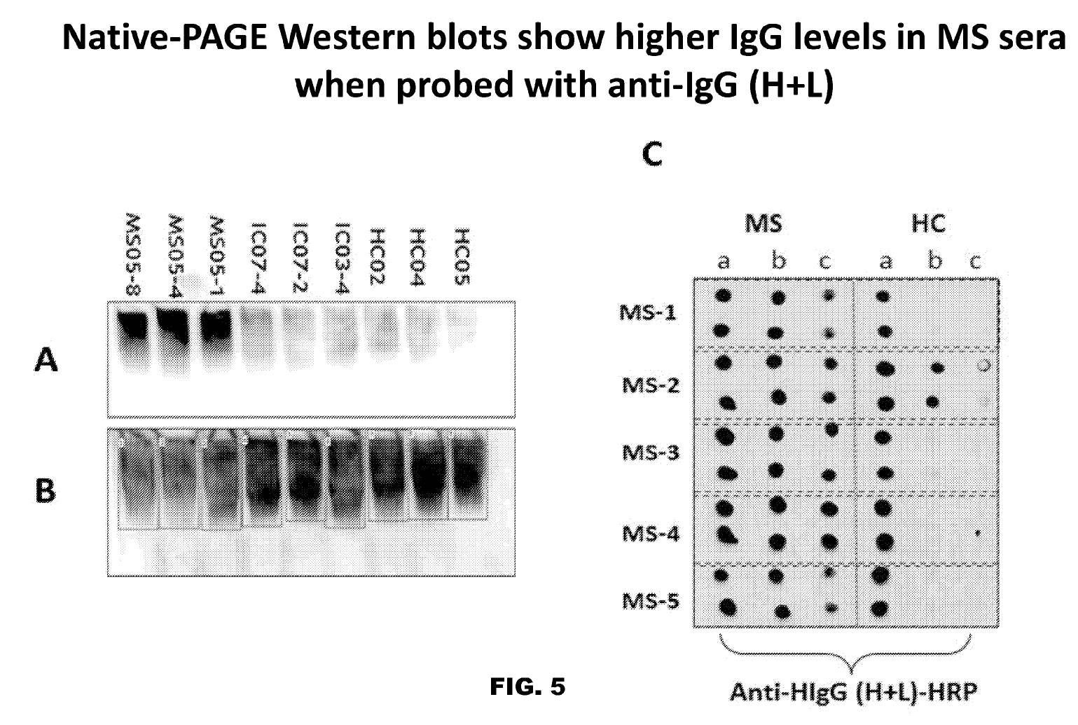

FIG. 5 (a) and 5 (b), show native-PAGE Western blots of MS sera compared to control. The MS sera shows elevated higher molecular weight IgG levels when probed with anti-IgG (H+L)-HRP (5a). Native polyacrylamide gel electrophoresis (4-15%) was run of MS, IC, and HC sera. Equal amounts of sera ( 3 μl of 1:10 dilution) were separated by a 4-15% Bio-Rad MINT PROTEAN TGX gel under non-reducing conditions. FIG. 5(a) shows a duplicate gel which was blotted and probed with anti-human IgG (H+L)-HRP, demonstrating the presence of large amounts high MW IgG in MS, but not in IC and HC samples. FIG. 5(b) shows a blot probed with anti-human IgG-Fc, showing less IgG in MS than that in IC and HC. Thus, according to this 3 MS, 3 IC, and 3 HC equal-volume serum analysis, MS patients on average contain 2.27× (2.27 fold) more IgG than inflammatory control patients and 3.73× (3.73 fold) more IgG than healthy controls when probing for IgG heavy and light chain components on a non-denatured polyacrylamide gel. FIG. 5(c) shows Dot Blots which demonstrate that serum IgG levels in MS are 1.6 times higher than that of controls. Representative Dot Blots showing 5 MS and 5 HC sera in 2-fold serial dilutions probed with anti-human IgG (H+L)-HRP. Equal amounts of sera MS and HC (5 μl in TBST) of 3 serial 2-fold dilutions in duplicate were spotted onto nitrocellulose membranes. Total IgG was detected with HRP-conjugated goat anti-human IgG (H+L) (1:10,000), followed by incubation with SuperSignal® West Pico Maximum Sensitivity chemiluminescent substrate.

### Example 3

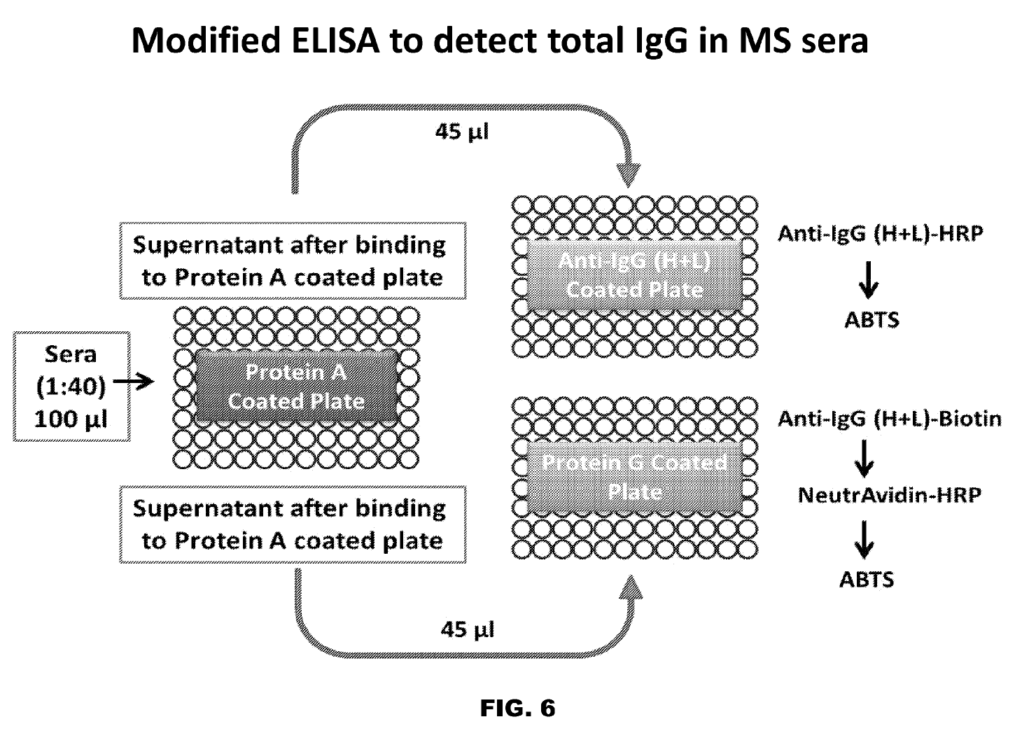

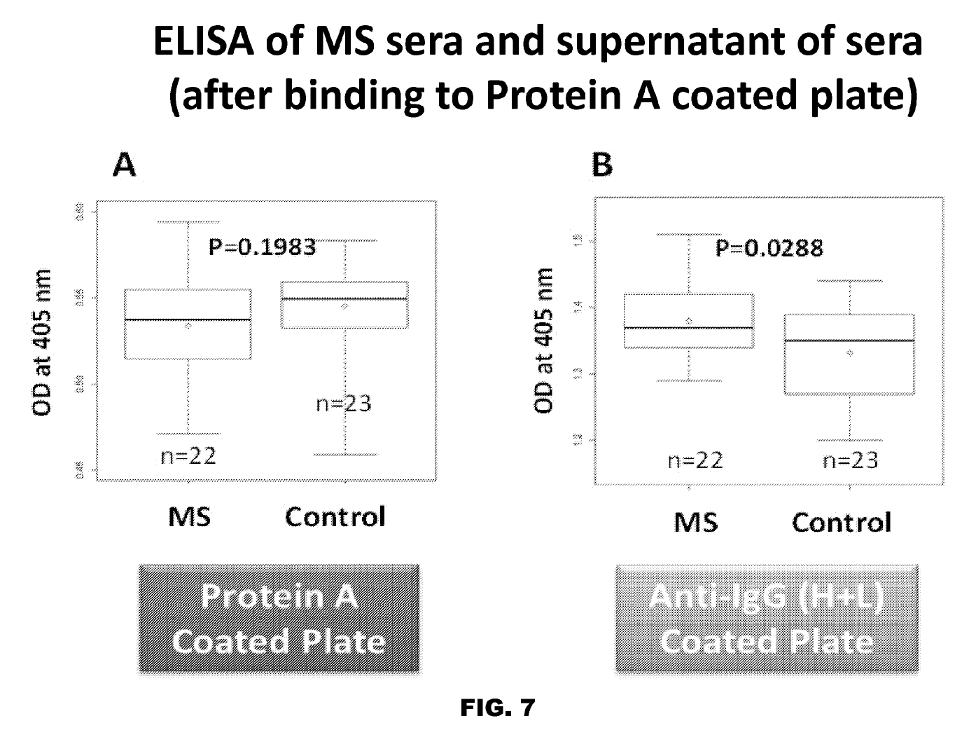

FIG. 6 shows a flow chart for an ELISA format for measuring IgG from sera. Total sera (1:40 dilution) in 100 μl TBS were added to Protein A coated microplates. After one hour incubation at room, half of the supernatants (45 μl) from each well were transferred into wells of ELISA plates pre-coated with anti-human IgG (H+L), and the other half of the supernatants (45 μl) were transferred to Protein G coated microplates. ELISA was carried out with all three plates. For samples in Protein A and Protein G coated plates, biotinylated goat anti-human IgG (H+L) antibody was used for detection, followed by addition of NeutrAvidin-HRP. For samples in anti-human IgG (H+L) coated plates, IgG was detected by addition of anti-human IgG (H+L)-HRP. ABTS substrate was used for color reaction. Optical density (OD) was measured with a microtiter plate reader at 405 nm. FIGS. 7-9 show the results.

FIGS. 7(a) and 7(b) show that higher IgG levels are only detected in the supernatant of MS sera after binding to a Protein A coated ELISA plate (Protein A flow through). FIG. 7(a) shows data from ELISA based format of FIG. 6. IgG levels are similar in both MS and control (p=0.1983) when original sera were coated directly onto Protein A ELISA plate. For Protein A flow through transferred to anti-human IgG (H+L) coated plates, there is a significant higher levels of IgG in the Protein A flow through of MS sera compared to controls (p=0.0288), indicating that the elevated IgG complexes are retained in the A-FT of MS sera.

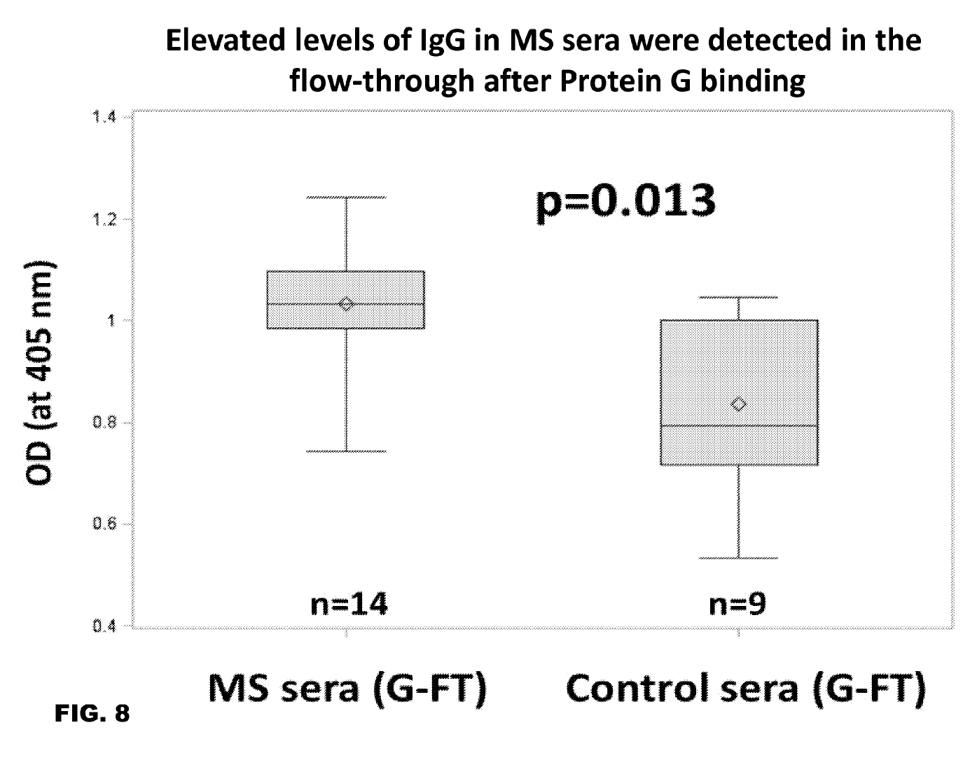

FIG. 8 shows that there were significantly elevated amounts of IgG antibodies present in the flow-through after Protein G purification of MS sera. We collected the flow-through after sufficient amounts of Dynabeads® Protein G Magnet beads (35 μl) binding to sera (2 μl) of MS and controls. Equal amounts of flow-through (5 μl) mixed with 95 μl of TBST were added to anti-human IgG (H+L) coated ELISA plate in triplicates. Bound IgG was detected with anti-human IgG (H+L)-HRP followed by addition of ABTS substrate. OD was measured with a micro titer plate reader at 405 nm. Higher mean OD values were observed in MS than controls (p=0.013).

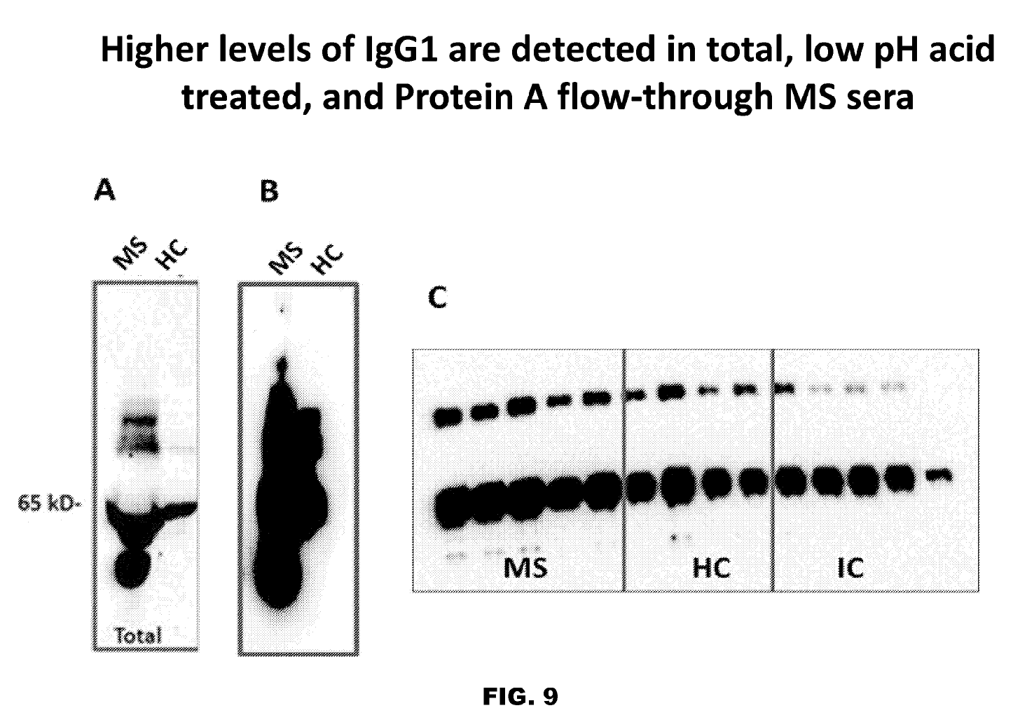

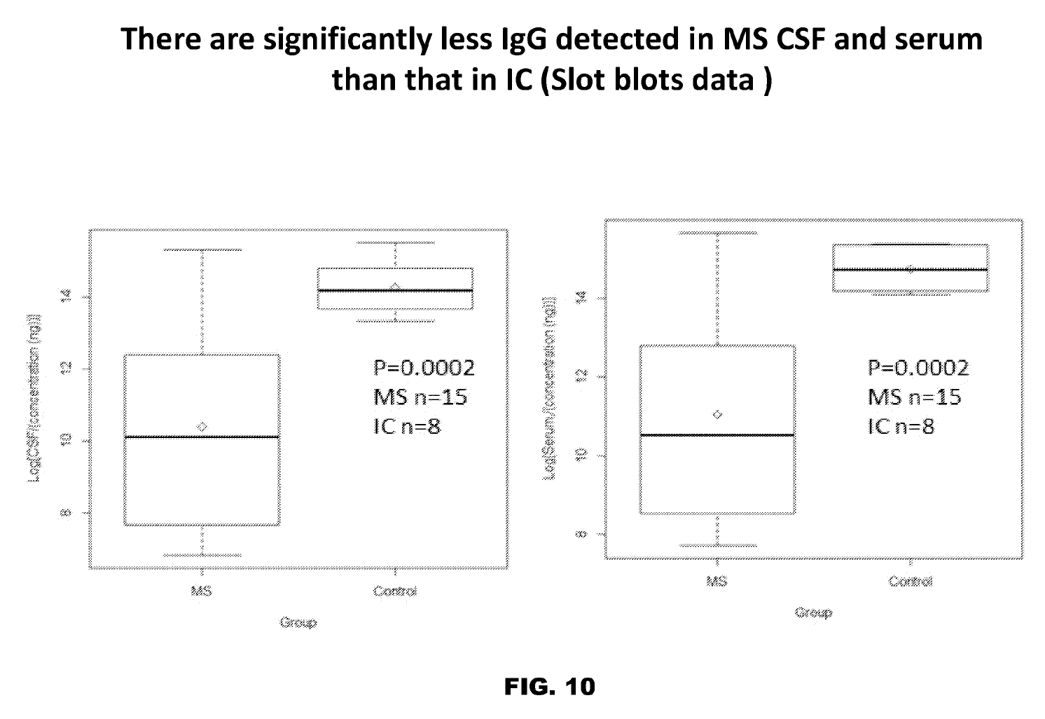

FIG. 9(a), 9(b), and 9(c) show that higher levels of IgG1 are detected in total, low pH acid treated, and Protein A flow-through MS sera. SDS-PAGE Western blots were probed with anti-human IgG1. Elevated levels of IgG1 in total MS serum (FIG. 9(a)) were detected. Glycine buffer (0.1 M, pH 2.5) was added to sera samples and incubated at room temperature for 30 min to generate acid treated sera (FIG. 9(b)). We collected flow through (supernatant) after Protein A binding of serum, and analyzed the presence of IgG1 and IgG3 with SDS-PAGE Western blots. We demonstrated that the flow through after both Protein A binding of MS CSF retained higher amounts of IgG1 (FIG. 9(c)) and IgG3 (data not shown). FIG. 10 shows that under nondenaturing conditions, there are significantly less IgG detected in MS CSF and serum than that in inflammatory CNS controls (IC). Slot blots of CSF and sera performed under non-denaturing conditions and probed with anti-human IgG (H+L)-HRP show significantly lower levels of IgG in MS CSF and serum (n=15) than IC (n=8), p=0.0002. These results indicate the presence of IgG complexes whose IgG are not detected under non-denaturing condition. Data summary of CSF IgG are shown on the left and serum IgG on the right.

### Example 4

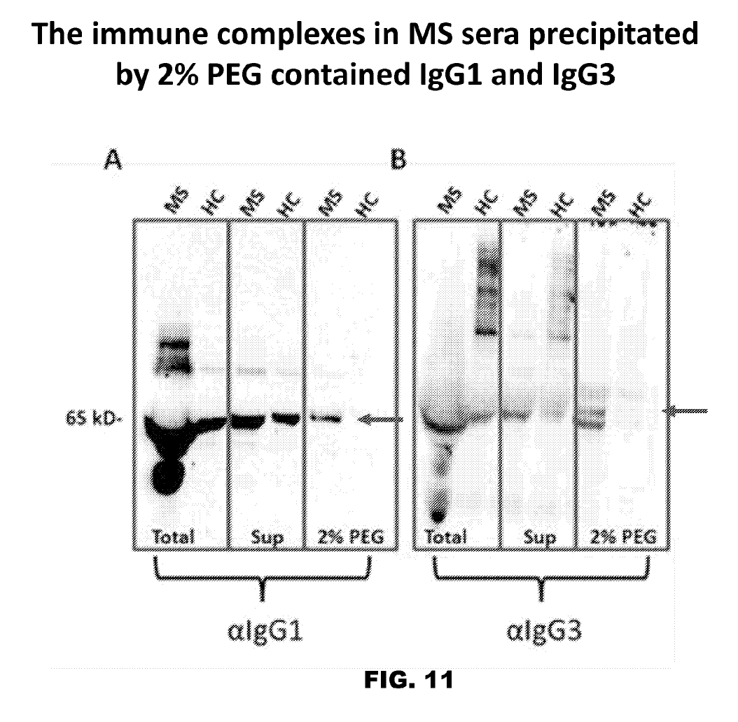

Treating sera with 2% PEG to precipitate protein complexes can selectively precipitate immune complexes. FIG. 11(a) and 11(b) indicate that IgG3-IgG1 immune complexes in MS serum can be precipitated by 2% PEG-8000; FIG. 11 shows SDS-PAGE Western blots of 2% PEG precipitates of MS serum and HC, probed with anti-IgG1 (FIG. 11(a) and IgG3 antibodies (FIG. 11(b)). The IgG3-IgG1 complexes in MS are recognized by anti-IgG1 (left) and IgG3 antibodies (right). Sup, supernatant after 2% PEG. Notice that most of the IgG3 in MS are shown as a 65 kD band in total, sup and PEG, but IgG3 are shown as high MW bands in HC.

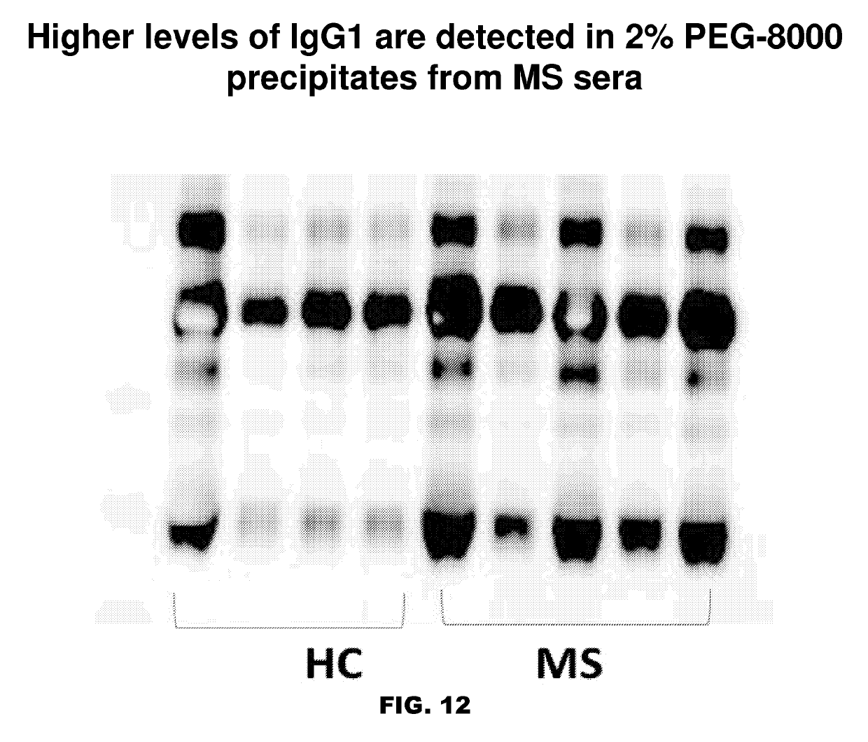

After treating MS sera with 2% PEG-8000, higher levels of IgG1 are detected in MS sera than in HC sera. FIG. 12 shows SDS PAGE Western probed with anti-human IgG1 of 2% PEG precipitates. Equal amounts of sera from MS and HC were mixed with PEG-8000 to reach final concentration of 2% PEG. After incubation at room temperature for 30 min, the mixtures were spun down and the pellets were dissolved in 10 mM Borate buffer prior to SDS PAGE.

### Example 5

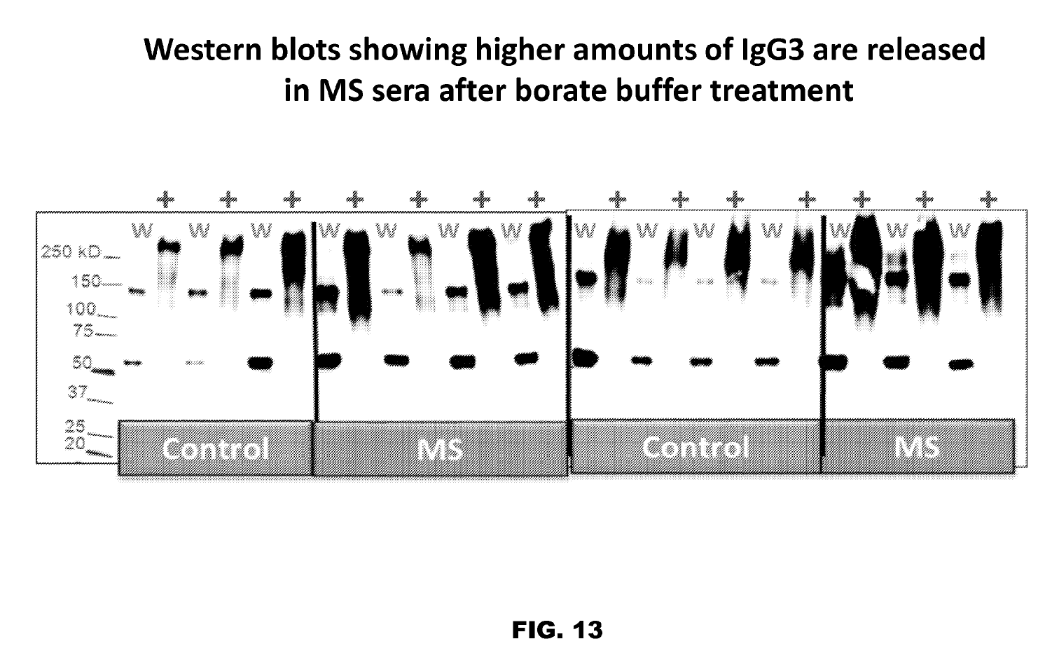

FIG. 13 shows SDS PAGE Western blots which indicate that higher amounts of IgG3 are released in MS sera after borate buffer treatment. Samples treated with water (W) and Borate buffer (+) as in FIG. 3 and 4 in parallel were carried out in SDS-PAGE Western blots and probed with anti-human IgG3. Notice IgG3 levels in higher molecular weight range (>100 kD) are much higher in the borate buffer treated MS sera compared to controls (IC, NIC and HC). In addition, water treated MS sera showed increased levels of 150 kD band compared to controls. On average, MS sera contain 2.5 times higher amounts of IgG3 than the control sera (by band intensity analysis.) Plus sign indicates samples treated with Borate buffer, and W indicated water treatment.

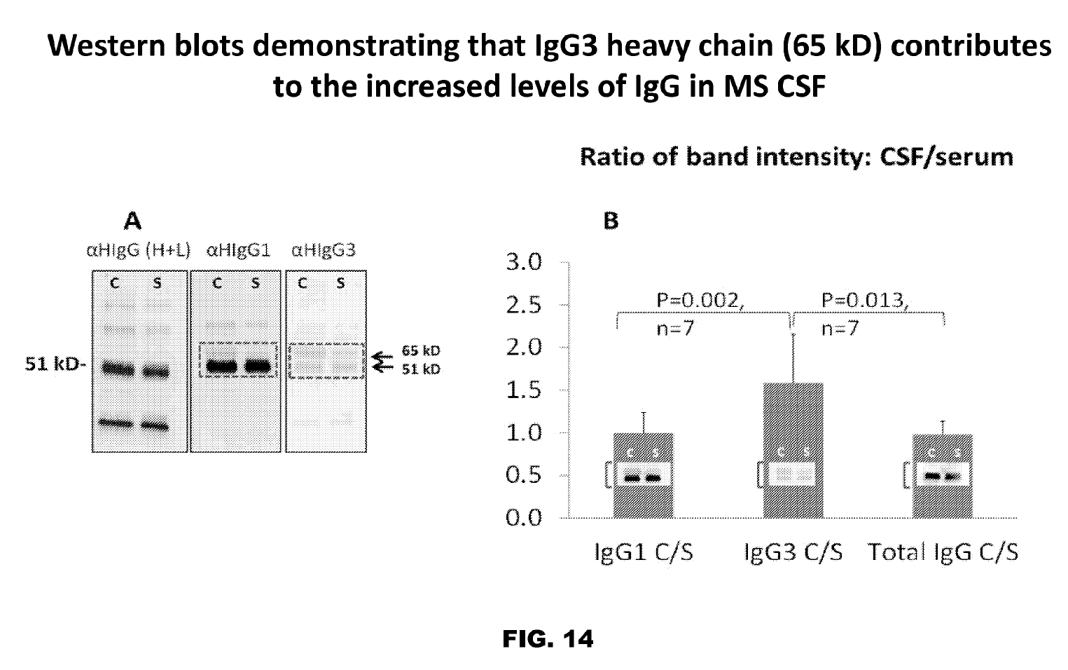

Western blot demonstrates that IgG3 heavy chain contributes to the increased levels of IgG in MS CSF. FIG. 14(a) shows total MS CSF and serum (1 μIgG/well) SDS PAGE Western blots probed with anti-human IgG (H+L)-HRP (left), anti-IgG1 (middle), and anti-IgG3 (right), followed by secondary antibodies and chemiluminescent detection. We further carried out band intensity analysis using the highlighted area of FIG. 14(a). FIG. 14(b) shows that the ratio of IgG3-reactive antibodies between CSF and serum was significantly higher (1.58) compared to the CSF/serum ratios of IgG1 (0.995) and total IgG (0.979), indicating that the IgG3 antibody contributes to the increased IgG in MS CSF.

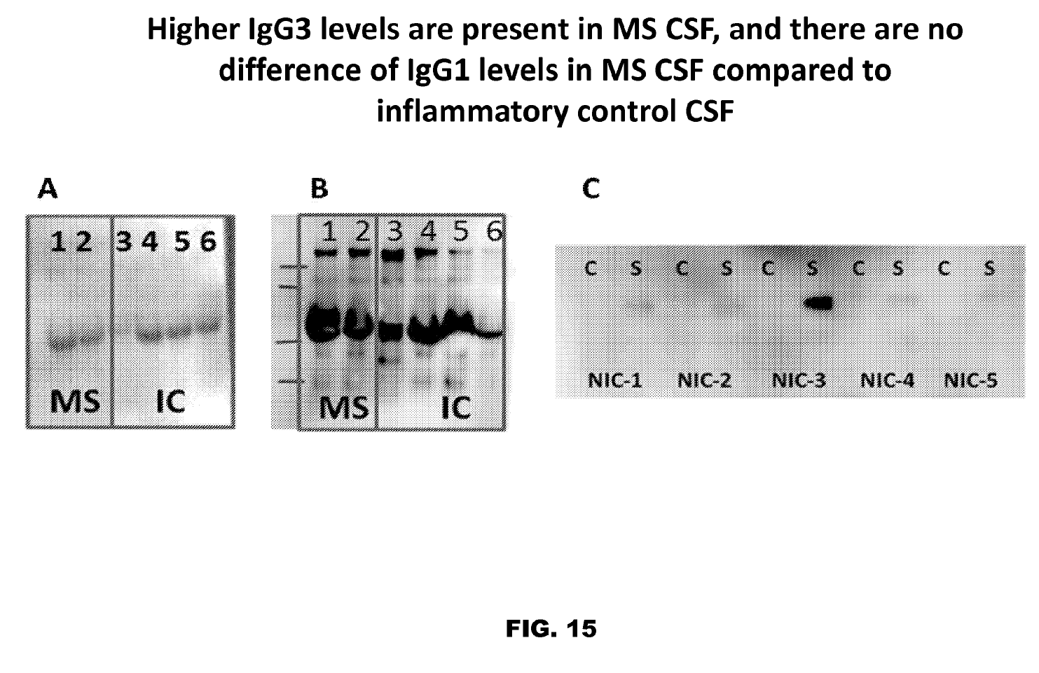

SDS PAGE Western blotting indicates that higher IgG3 levels are present in MS CSF, and that there are no differences of IgG1 levels between MS CSF and inflammatory control CSF. SDS-PAGE (Bolt Bis-Tris Plus Gel 4-12%) Western blots of MS and IC CSF were blotted onto PVDF membrane, followed by detection with rabbit anti-human IgG1 or IgG3 as primary antibodies and anti-rabbit-HRP as secondary antibody. FIG. 15(a) shows the blot probed with mouse anti-human IgG1 antibody. FIG. 15(b) shows the same blot that was stripped, and re-probed with anti-human IgG3 antibody. Lane 1. SPMS; Lane 2. RRMS; Lane 3. IC 06-1 (SSPE); Lane 4. IC 07-2 (chronic progressive meningoencephalitis); Lane 5. IC 06-5 (paraneoplastic encephalitis); and Lane 6. IC 05-2 (Chronic meningitis). FIG. 15(c) shows that no IgG3 are detected in the CSF of patients with non-inflammatory CNS diseases. Western blots of paired CSF and serum probed with anti-IgG3 antibody show no IgG are present in the CSF of NIC.

### Example 6

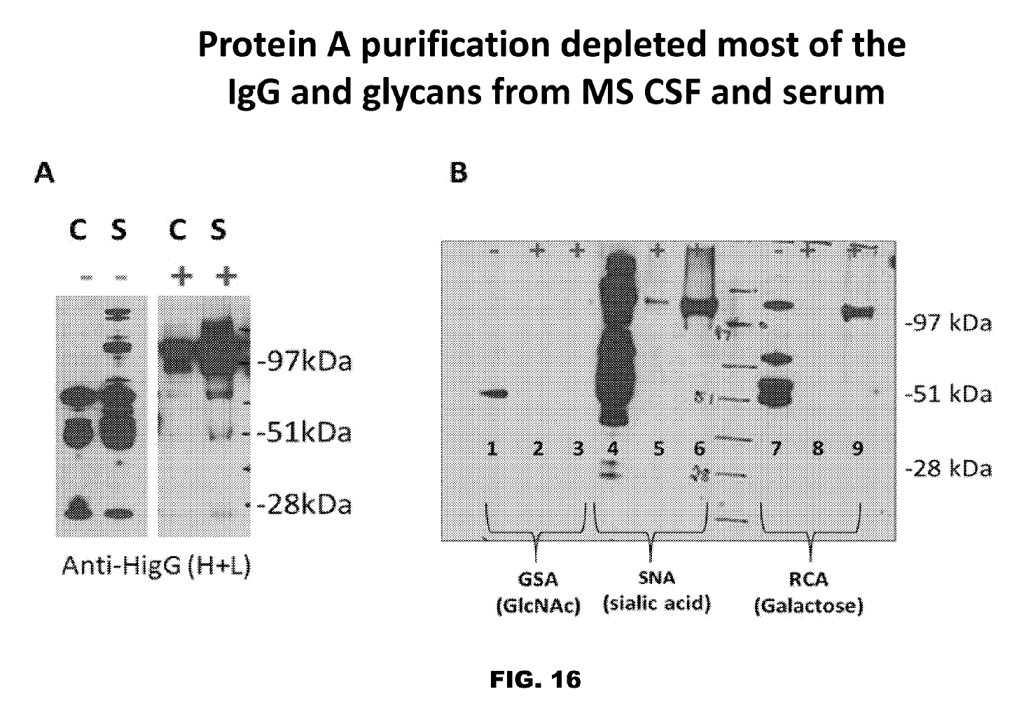

Protein A purification depleted most of the IgG heavy and light chains and glycans in MS CSF and serum. FIG. 16(a) shows SDS-PAGE Western blots of original and purified IgG by Protein A Dynabeads. Equal amounts of total serum without Protein A purification (−) and Protein A purified IgG (+) were separated by SDS-PAGE, blotted and probed with anti-human IgG (H+L). FIG. 16(b) same conditions shows probe by the three most common biotinylated lectins for determining the specificity of respective sugars on the IgG antibodies. Vector Elite ABC Vectastain was used for signal detection. Lane 1: Original MS Serum #14 (500 ng); Lane 2: Purified IgG (100 ng); Lane 3: Purified IgG (500 ng); Lanes 4-6 and Lanes 8-10, same as Lanes 1-3 but Probed with SNA or RCA. GSA (Griffonia simplicfolia Lectin II) recognizes exclusively alpha-or beta-linked N-acetylglucosamine (GlcNAc) residues on the non-reducing terminal of oligosaccharides. SNA (Sambucus nigra Lectin (SNA) binds preferentially to sialic acid attached to terminal galactose in (alpha-2,6). RCA (Ricinus communis Agglutin I (RCA) detects oligosaccharides ending in galactose.

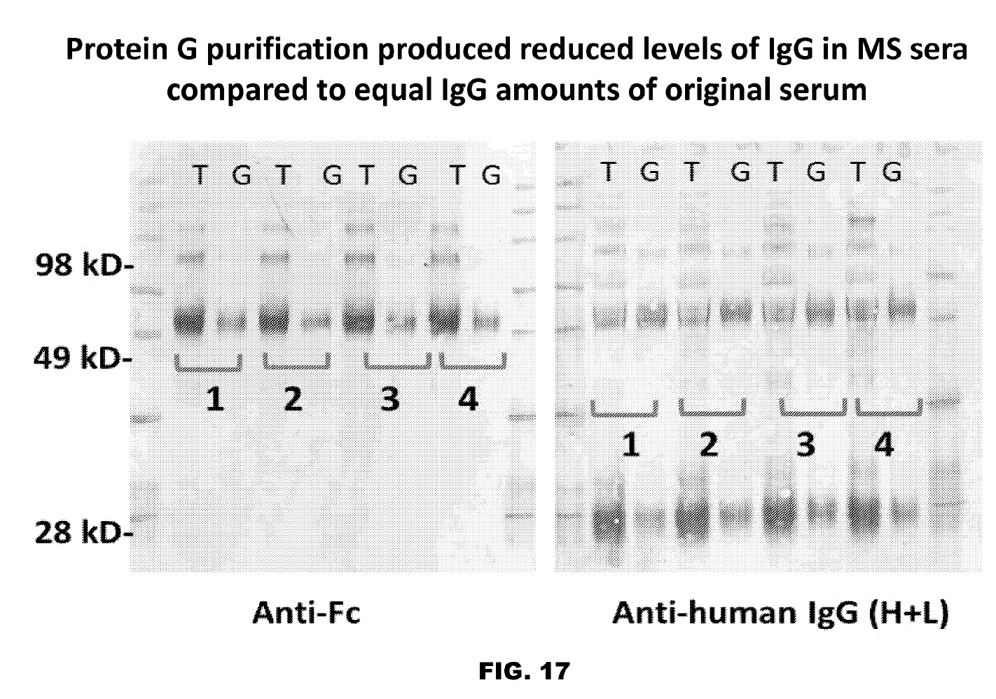

FIG. 17(a) and 17(b) indicate that Protein G purified MS sera show reduced amounts of heavy chain (detected by anti-human Fc-AP) and light chain (detected by anti-human IgG (H+L)-AP IgG compared to the original serum. FIG. 17(a) shows Western blots of MS original and Protein G purified serum IgG (paired) probed with anti-human IgG Fc specific antibody-AP; FIG. 17(b) shows Western blots of MS original and Protein G purified serum IgG (paired) probed with anti-human IgG (H+L)-AP. T: total serum; G: Protein G purified IgG. Number 1-4 represent 4 MS patients. These results indicate that Protein G could not completely access to the Fc region and some of the light chain of the IgG immune complexes and produced less IgG heavy chain and light chain.

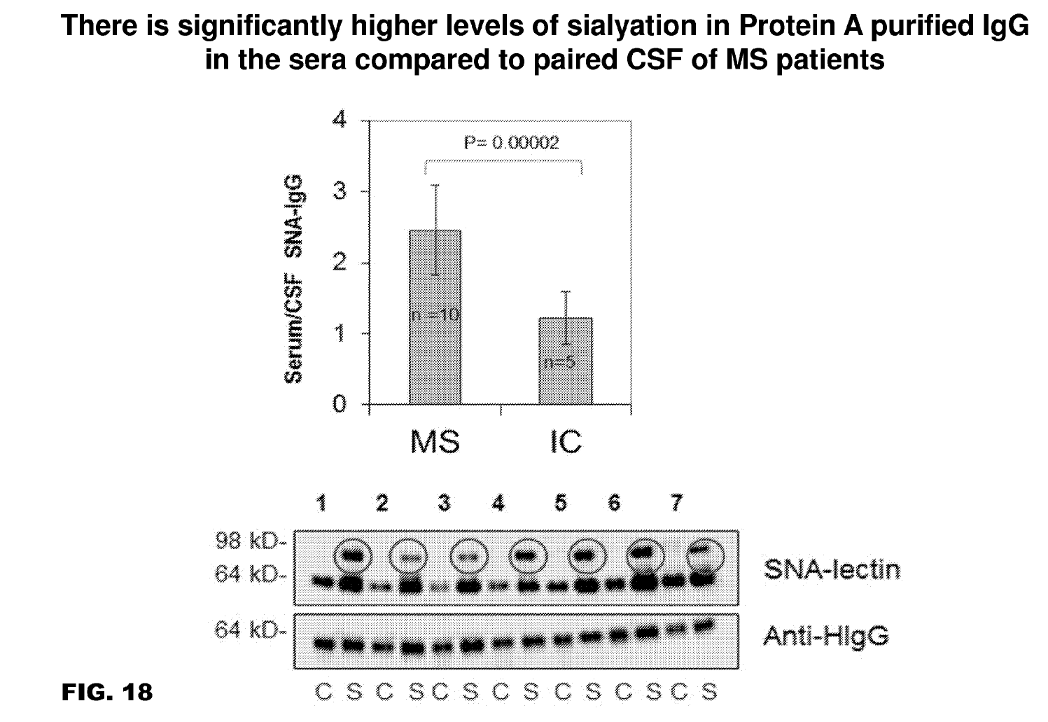

FIG. 18 shows that there is a significant decrease of sialylated IgG Fc in the CSF compared to paired serum in MS. We purified IgG antibody in CSF and paired serum from 12 MS patients and 9 inflammatory CNS controls with Protein A Magnetic Beads. The purified IgG were quantified for sialic acid level using immunoblotting probed with sialic acid specific lectin SNA. There was a significant decrease of sialylated IgG Fc (the 64 kD band ) in the CSF compared to paired serum (p=6.2) in MS. In contrast, no significant difference of IgG sialylation was found in the CSF and serum of CNS inflammatory controls (p=0.18). These results suggest that most of glycosylated IgG in CSF are formed IgG immune complexes and could not be purified by Protein A, further confirm that the intrathecal IgG in MS CSF are contributed by glycosylated IgG immune complexes which could not be purified well by Protein A.

### Example 7

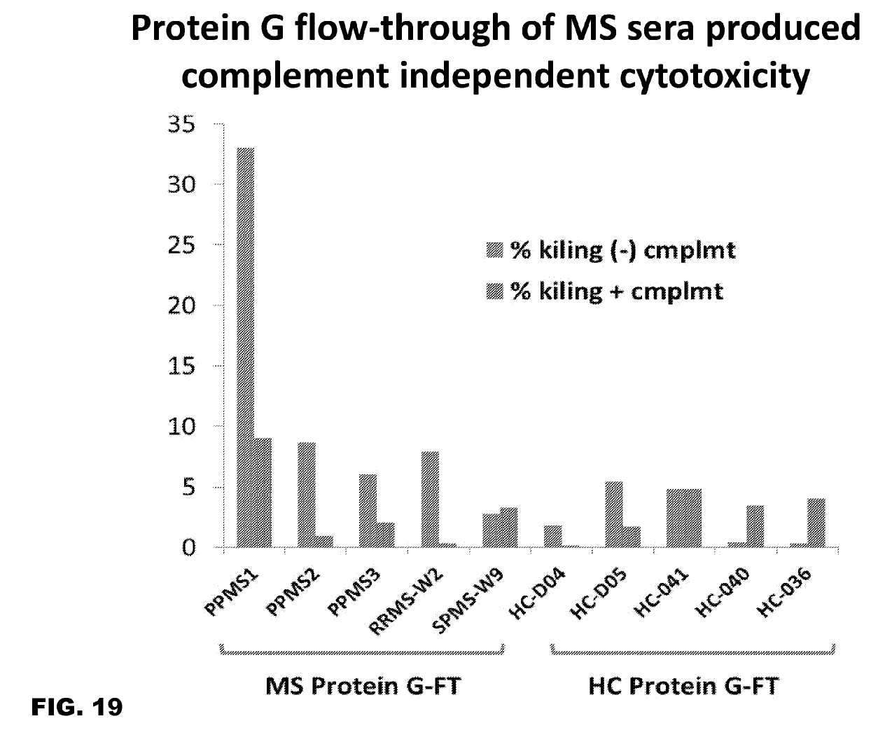

FIG. 19 shows the complement independent cytotoxicity of MS sera G-FT on mouse CNS cells. IgG3-IgG1 complexes from Protein G flow-through was found to display cytotoxicity on murine neuronal cells without addition of complement. IgG3-IgG1 complexes were obtained from the sera of MS patients or healthy controls following Protein G purification. Primary mouse neonatal cells were cultured under standard conditions, and were treated with Protein G-FT (IgG complexes). Medium was harvested over a time course and assayed for lactate dehydrogenase (LDH). Significantly higher levels of cytotoxicity was observed in MS G-FT in samples without addition of complement.

### Example 8

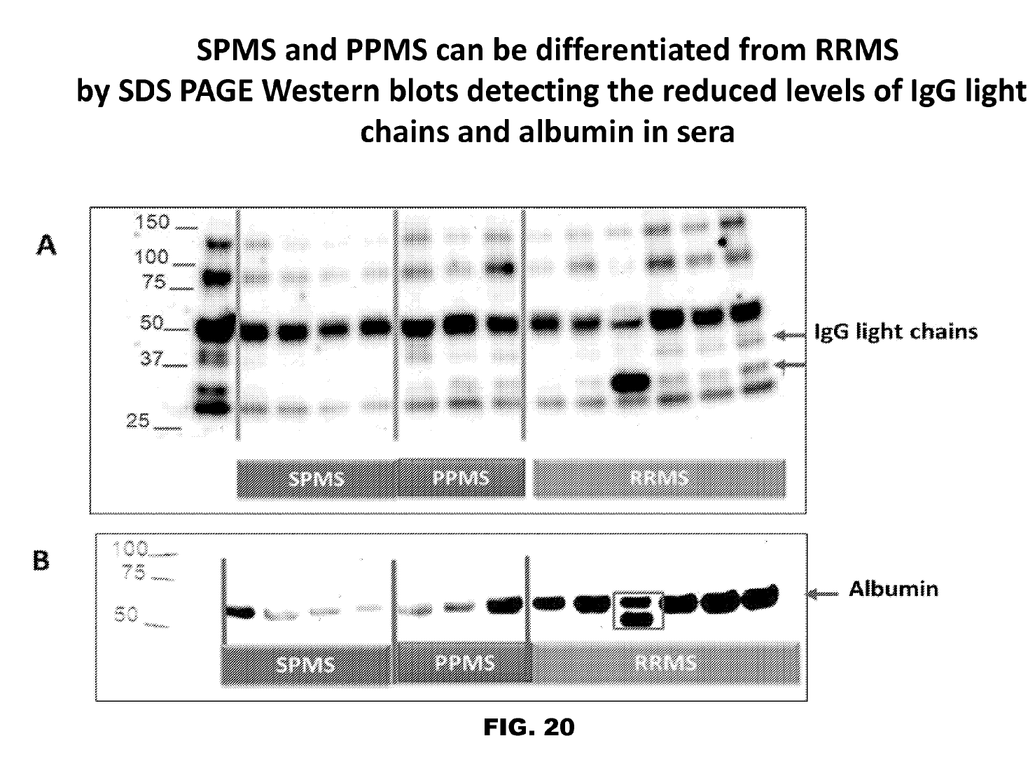

FIG. 20(a) and (b) shows that SPMS and PPMS can be differentiated from RRMS by reduced levels of IgG light chains and albumin. FIG. 20(a) shows a Western Blot probed with Total IgG (H+L) probe indicating that SPMS have lower levels of IgG light chains (between 25 kD-50 kD). FIG. 20(b) shows that Western Blot of MS sera probed with anti-human albumin demonstrating reduced levels of albumin in both SPMS and PPMS compared to RRMS.

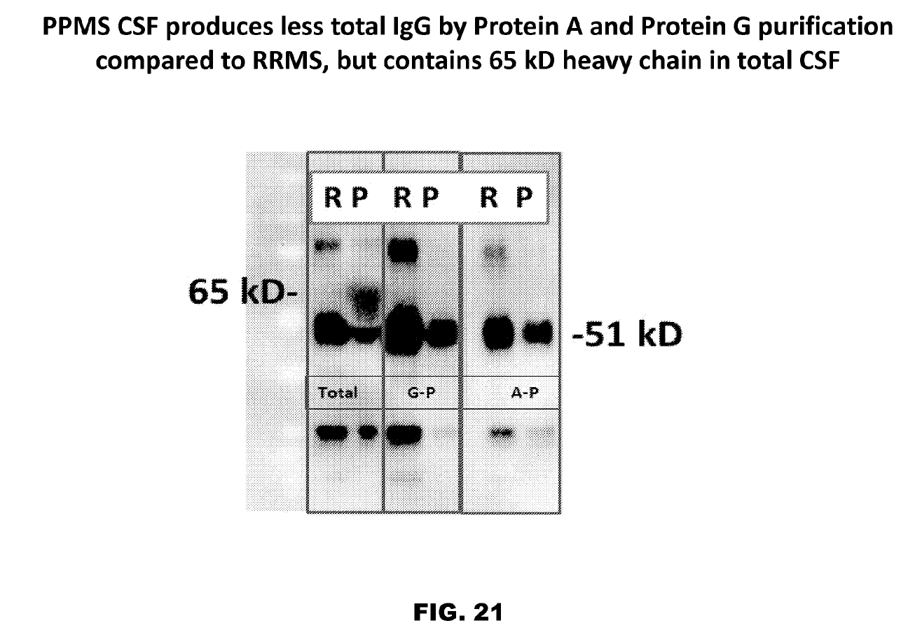

FIG. 21 shows that PPMS CSF (P) produce less total IgG (51 kD heavy chain and 25 kD light chain) by Protein A and Protein G purification compared to RRMS CSF (R), but contain 65 kD heavy chain in total CSF, as shown by Western Blot probed with anti-human IgG (H+L) antibody.

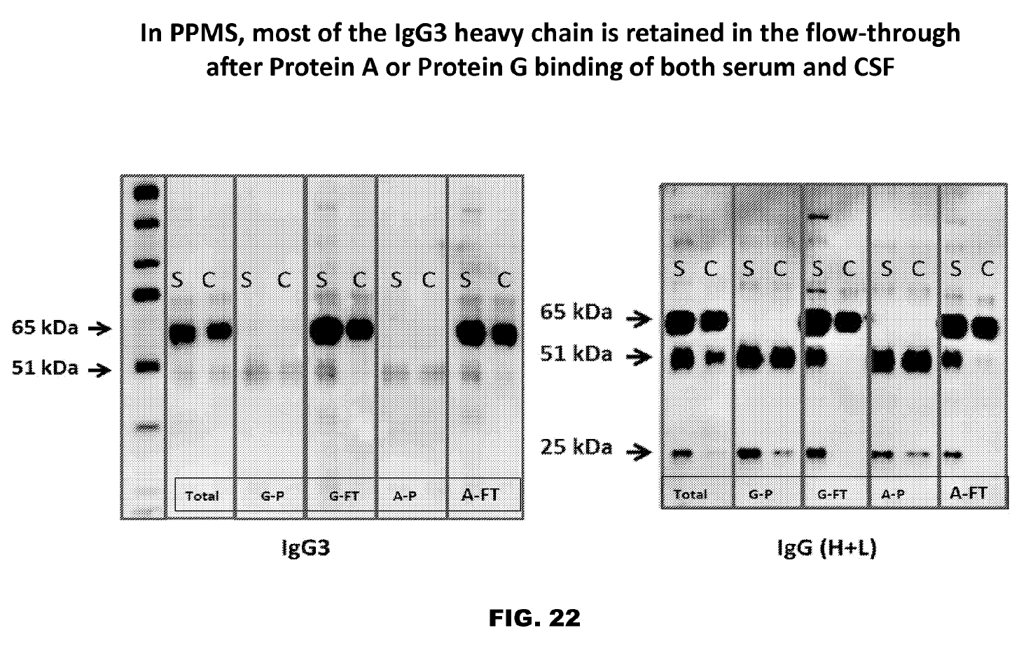

FIG. 22 shows that in PPMS (PPMS1), most of the IgG3 heavy chain (65-kD) is retained (present) in the flow-through after Protein A or Protein G binding in both serum and CSF. Western blots of various sample preparations from CSF and serum of PPMS1 probed with anti-IgG3 (left) or anti-human IgG (H+L) antibodies (right). Total CSF (7 μg) and serum (2 μl) in 200 μl PBS Tween were incubated with 35 μl of Protein A or Protein G Magnetic beads at RT for 1 h, the supernatant (FT) was collected after bead binding. L: molecular weight marker; Total: total serum and CSF (1 μg/well); G-P: Protein G purified IgG (1 μg/well); G-FT: flow-through after Protein G purification ( 1/40 of the total flow-through); A-P: Protein A purified IgG (1 μg/well); A-FT: flow-through after Protein A purification ( 1/40 of the total flow-through).

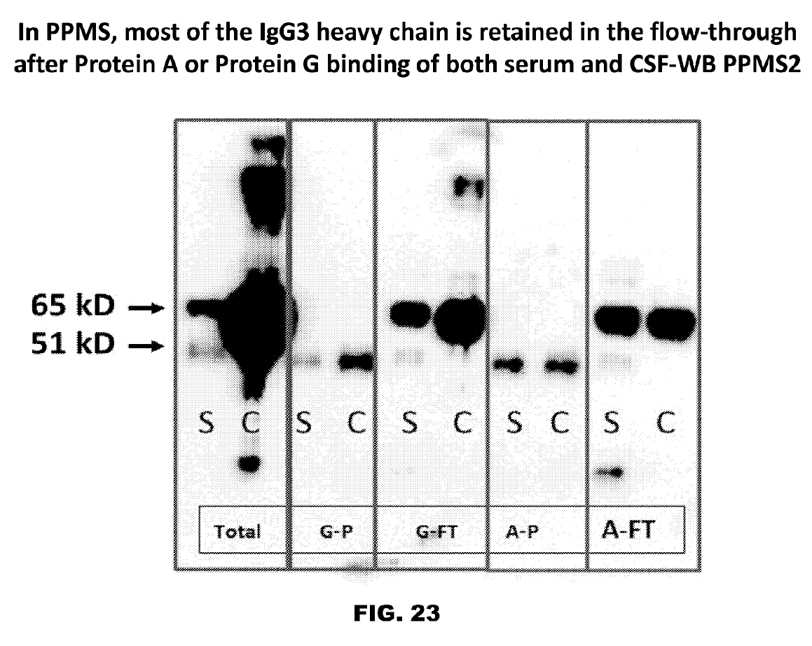

FIG. 23 shows SDS-PAGE Western blots of PPMS2, demonstrating again that most of the IgG3 heavy chain (65-kD) is retained (present) in the flow-through after Protein A or Protein G binding in both serum and CSF. The blots were probed with anti-human IgG (H+L).

The description of the various embodiments has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiments described and shown in the FIGS. were chosen and described in order to explain the principles of the invention, the practical application, and to enable others of ordinary skill in the art to understand the invention for various embodiments with various modifications as are suited to the particular use contemplated. All references cited herein are incorporated in their entirety by reference.

What is claimed is:

1. A method to detect whether a patient has an elevated level of OqG immune complexes or at least one protein comprising:
a. obtaining a sample from the patient either having or at risk of having multiple sclerosis (MS);
b. treating the sample to dissociate immune complexes of IgG₁ and IgG₃ or the at least one protein;
c. contacting the sample from the patient with a reagent that binds specifically to a human IgG or the at least one protein;
d. comparing the amount of IgG or the amount of the at least one protein in the sample compared to a control sample; and
e. determining whether the patient has an elevated level of human IgG or the at least one protein compared to the control.

2. The method of claim 1, wherein treating step (b) comprises
i. contacting the sample with at least one affinity resin selected from the group consisting of Protein G, Protein A, Protein A/G, anti-human IgG-Fc specific, or a combination thereof; and
ii. separating the nonbound fraction thereof from the affinity resin-, wherein the nonbound fraction is the sample and is treated to dissociate immune complexes of IgG₁ and IgG₃ or the at least one protein.

3. The method of claim 1, wherein treating step (b) comprises contacting the sample from the patient with a polyethylene glycol to selectively precipitate immune complexes of IgG₁ and IgG₃ or the at least one protein and collecting the precipitate wherein the precipitate is the sample and is treated to dissociate immune complexes of IgG₁ and IgG₃ or the at least one protein.

4. The method of claim 3, wherein the polyethylene glycol is PEG-8000.

5. The method of claim 1, wherein the reagent which binds specifically to a human IgG is selected from the group consisting of one or more antibodies, a fragment thereof, and a biotinylated lectin.

6. The method of claim 5, wherein the antibody or fragment thereof is selected from the group consisting of an antibody or fragment thereof which binds selectively to a whole human IgG (heavy chain and light chain), a human IgG heavy chain, a human IgG light chain, human IgG subclass IgG₁, human IgG subclass lgG₃, human Fc, and a human glycosylated IgG.

7. The method of claim1 wherein the contacting steps employs a technique selected from the group consisting of an ELISA, dot blots/slot blots, and a Western blot, and the elevated level of IgG is detected by increased level of IgG relative to the control.

8. The method of claim 1, wherein the dissociation method comprises an acidic treatment, a basic treatment, a heat treatment, a treatment to lower surface tension, or combinations thereof.

9. The method of claim 8, wherein the basic treatment is treatment with a borate buffer at a pH of above 10.

10. The method of claim 1, wherein the sample is a serum sample.

11. The method of claim 1, wherein the sample is CSF.

12. (canceled)

13. The method of claim 1, wherein the at least one protein is selected from the group comprising total protein, albumin, total IgG, IgG₃, IgG₁, glycosylated IgG gllycosylated IgG₃ and glycosylated IgG₁.

14.-19. (canceled)

20. The method of claim 13, wherein the glycosylated IgG₃ is O-linked glycosylated IgG₃.

21.-25. (canceled)

26. The method of claim 1, wherein the control sample is an IgG complex or at least one protein obtained from human serum or CSF sample obtained from a healthy donor, a healthy donor pool a patient having an Inflammatory or non-inflammatory CNS condition, or a patient pool having an inflammatory or non-inflammatory CNS condition.

27.-37. (canceled)

38. A method to determine efficacy of an MS drug or drug candidate in a patient, comprising:
a. obtaining a sample from the patient having been treated with the drug or drug candidate;
b. treating the sample to dissociate immune complexes of IgG₁ and IgG₃ or at least one protein.
c. contacting the sample from the patient with a reagent which binds specifically to a human IgG or the at least one protein;
d. comparing the amount of human IgG or the at least one protein in the sample with a control sample; and
e. determining whether the patient has a decreased level of IgG or the at least one protein compared to the control, wherein the decreased levels indicate efficacy for the MS drug or drug candidate.

39. The method of claim 38, wherein the reagent that binds specifically to human IgG is selected from group consisting of one or more antibodies, a fragment thereof, and a biotinylated lectin.

40. (canceled)

41. The method of claim 38, wherein treating step (b) comprises
i. contacting the sample with at least one affinity resin selected from the group consisting of Protein G, Protein A, Protein A/G or a combination thereof; and
ii. separating the nonbound fraction thereof from the affinity resin, wherein the nonbound fraction is the sample and is treated to dissociate immune complexes of IgG₁ and IgG₃ or the at least one protein.

42. The method of claim 38, wherein step (b), comprises contacting the sample from the patient with a polyethylene glycol to selectively precipitate immune complexes of IgG₁ and IgG₃, and collecting the precipitate, wherein the precipitate is the sample and is treated to dissociate immune complexes ot IgG₁ and IgG₃ or the at least one protein.

43.-55. (canceled)

56. The method of claim 1, wherein step (e) comprises determining whether the patient has a level of human IgG immune complex or the at least one protein that is comparable to a control sample, wherein the control sample is a sample from a patient pool having primary-progressive MS (PPMS) or a patient pool having relapsing-remitting MS (RRMS).

57. The method of claim 1, further comprising e. administering an effective amount of a pharmaceutical compound that is effective to treat MS to the patient having elevated levels of IgG or the at least one protein.
